(19) United States
(12) Patent Application Publication
(10) Pub. No.: US 2024/0226117 A1
(43) Pub. Date: Jul. 11, 2024

(54) TREATMENT OF CONGENITAL ADRENAL HYPOPLASIA

(71) Applicant: ReveraGen BioPharma, Inc., Rockville, MD (US)

(72) Inventors: Eric HOFFMAN, Rockville, MD (US); Jesse DAMSKER, Alexandria, VA (US)

(21) Appl. No.: 18/607,927

(22) Filed: Mar. 18, 2024

Related U.S. Application Data
(63) Continuation of application No. PCT/US2022/076686, filed on Sep. 20, 2022.
(60) Provisional application No. 63/272,236, filed on Oct. 27, 2021.
(60) Provisional application No. 63/246,550, filed on Sep. 21, 2021.

(51) Int. Cl. A61K 31/573; A61K 9/00; A61K 9/10; A61K 47/02; A61K 47/10; A61K 47/12; A61K 47/14; A61K 47/26; A61K 47/36; A61K 47/38; A61P 21/00

(52) U.S. Cl. CPC A61K 31/573; A61K 9/0053; A61K 9/10; A61K 47/02; A61K 47/10; A61K 47/12; A61K 47/14; A61K 47/26; A61K 47/36; A61K 47/38; A61P 21/00

(57) ABSTRACT

The present disclosure provides a method of treating or reducing the symptoms of congenital adrenal hypoplasia in a human patient, comprising administering to the human patient in need thereof a therapeutically effective amount of vamorolone or a salt or polymorph thereof.

This application is a bypass continuation of International Application No. PCT/US2022/076686, filed Sep. 20, 2022, which claims the benefit of priority of U.S. Provisional Patent Application Ser. No. 63/246,550 filed Sep. 21, 2021, and U.S. Provisional Patent Application Ser. No. 63/272,236 filed Oct. 27, 2021, the disclosures of each are incorporated by reference in their entireties for all purposes.

This invention was made with government support under grant Number NS095423 awarded by the National Institutes of Health. The government has certain rights in the invention.

Compositions and methods for treating congenital adrenal hypoplasia are disclosed herein.

Congenital adrenal hyperplasia (CAH or X-linked adrenal hypoplasia congenita) is a group of autosomal recessive genetic disorders that result in little or no cortisol biosynthesis. Disease severity is correlated with genotype. Almost 300 pathogenic mutations in CYP21A2 are known, but genotyping individuals with CAH is complex due to gene duplications, deletions, and rearrangements within chromosome 6p21.3.

The most frequent form of the disease is 21-hydroxylase deficiency caused by mutations in the CYP21A2 gene located on chromosome 6p21, which accounts for approximately 95% of CAH cases. These mutations can range from complete loss of enzyme activity required for synthesis of cortisol in the adrenal cortex to a spectrum of partial loss, which results in disease severity that is a direct consequence of a specific mutation. This continuum of 21-hydroxylase deficiency has been broadly classified into salt-wasting and simple-virilizing forms, grouped as classical CAH, and the milder form known as non-classic CAH (NCCAH) or “late-onset” CAH, which is usually diagnosed in late childhood or early adulthood. Non-classic CAH patients are either homozygous or compound heterozygotes, often with a classical CAH allele. These patients have sufficient enzyme activity (>20-50% of normal) such that they do not have salt-wasting or cortisol deficiency and have normal genitalia at birth, and many remain asymptomatic throughout life. About 5% of patients have mutations in 11-beta-hydroxylase, 3-beta-hydroxysteroid dehydrogenase, 17-hydroxylase, or steroidogenic acute regulatory (StAR) deficiency.

Both genetic mutations result in congenital adrenal hyperplasia, cortisol deficiency and excessive adrenocorticotropic hormone (ACTH) production with overproduction of androgens. These patients require lifelong management with glucocorticoids and the attendant problems associated with such treatment. Accordingly, a significant need exists for treatment regimens to improve the health, well-being, quality of life, and to manage related disorders in patient with CAH.

Provided is a method of treating or reducing the symptoms of congenital adrenal hypoplasia in a human patient, comprising administering to the human patient in need thereof a therapeutically effective amount of a compound having the structural formula

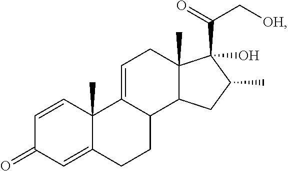 [SMILES: C[C@@H]1CC2C3CCC4=CC(=O)C=C[C@]4(C)C3=CC[C@]2(C)[C@@]1(O)C(=O)CO]

or a salt or polymorph thereof.

Other aspects of the invention disclosed herein will be set forth in greater detail as the patent disclosure proceeds.

### BRIEF DESCRIPTION OF THE DRAWINGS

FIG. 1 shows a flow diagram for the manufacturing process used to make the aqueous oral pharmaceutical suspension composition comprising vamorolone Form I described in Example 3.

FIG. 2 shows a flow diagram for the manufacturing process used to make the aqueous oral pharmaceutical suspension composition comprising vamorolone Form I described in Example 4.

### DETAILED DESCRIPTION

### Definitions

As used in the present specification, the following words and phrases are generally intended to have the meanings as set forth below, except to the extent that the context in which they are used indicates otherwise.

“Adrenocorticotropic hormone,” “ACTH,” “adrenocorticotropin,” or “corticotropin” is a polypeptide tropic hormone produced by and secreted by the anterior pituitary gland. ACTH is a component of the hypothalamic-pituitary-adrenal axis and is often produced in response to biological stress along with its precursor corticotropin-releasing hormone from the hypothalamus. Its principal effects are increased production and release of cortisol by the cortex of the adrenal gland. ACTH is also related to the circadian rhythm in many organisms.

Deficiency of ACTH indicates secondary or tertiary adrenal insufficiency. Secondary adrenal insufficiency is the suppressed production of ACTH due to an impairment of the pituitary gland or hypothalamus, such as hypopituitarism. Tertiary adrenal insufficiency is a disease of the hypothalamus, which decreases corticotropin-releasing hormone (CRH). Conversely, chronically elevated ACTH levels occur in primary adrenal insufficiency (e.g., Addison's disease) when adrenal gland production of cortisol is chronically deficient. In Cushing's disease, a pituitary tumor causes elevated ACTH from the anterior pituitary and an excess of cortisol, leading to hypercortisolism. This constellation of signs and symptoms is known as “Cushing's syndrome.”

The symptoms of adrenal insufficiency are weakness, fatigue, and loss of appetite. The first-line treatment for adrenal insufficiency is corticosteroids.

In certain embodiments, DMD can have adrenal insufficiency as part of the disease. DMD is a multi-organ syndrome that can express an array of symptoms. Also, the major adrenal insufficiency gene, DAX1, is neighboring the DMD gene at Xp21 and sometimes shows up as contiguous gene deletion syndrome.

As used herein, “vamorolone” refers to 17α,21-dihydroxy-16α-methylpregna-1,4,9(11)-triene-3,20-dione (also known as VBP15 or VB-15) and has the structure:

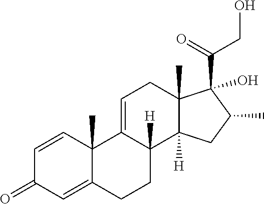

Vamorolone potently binds to the glucocorticoid receptor and has anti-inflammatory effects similar to traditional glucocorticoid drugs such as prednisone and deflazacort. Vamorolone lacks an 11-carbon oxygen group (hydroxyl or carbonyl) that is one of five molecular contact sites with the glucocorticoid receptor. In-vitro pharmacology and pre-clinical in vivo studies have shown that vamorolone retains the anti-inflammatory activity of steroid drugs while lacking the adverse effects (AEs) for these drugs, including stunting of growth, bone morbidities, and muscle atrophy, in these models. Many corticosteroids, including prednisone and deflazacort, are agonists of the mineralocorticoid receptor, leading to increased blood volume and pressure via the renin-angiotensin pathway. In contrast, vamorolone is a potent antagonist of the mineralocorticoid receptor, similar in activity to eplerenone and spironolactone. The differential mechanism of action of vamorolone compared to traditional corticosteroid anti-inflammatory drugs is attributed to the loss of gene transcriptional activities for glucocorticoid response element-binding and activation, potent antagonist activity for the mineralocorticoid receptor, superior membrane stabilization properties, and retention of the distinct NF-κB inhibitory (anti-inflammatory) activities.

Vamorolone can exist as various polymorphic forms. As used herein, the terms “polymorphs” and “polymorphic forms” and related terms herein refer to crystalline forms of the same molecule. Different polymorphs may have different physical properties such as, for example, melting temperatures, heats of fusion, solubilities, dissolution rates, and/or vibrational spectra because of the arrangement or conformation of the molecules in the crystal lattice. The differences in physical properties exhibited by polymorphs affect pharmaceutical parameters such as storage stability, compressibility and density (important in formulation and product manufacturing), and dissolution rates (an important factor in bioavailability). Differences in stability can also result from changes in chemical reactivity (e.g., differential oxidation, such that a dosage form discolors more rapidly when comprised of one polymorph than when comprised of another polymorph) or mechanical property (e.g., tablets crumble on storage as a kinetically favored polymorph converts to thermodynamically more stable polymorph) or both (e.g., tablets of one polymorph are more susceptible to breakdown at high humidity). As a result of solubility/dissolution differences, in the extreme case, some polymorphic transitions may result in a lack of potency or, at the other extreme, toxicity. In addition, the physical properties of the crystal may be important in processing; for example, one polymorph might be more likely to form solvates or might be difficult to filter and wash free of impurities (i.e., particle shape and size distribution might be different between polymorphs).

Polymorphs of a molecule can be obtained by several methods, as known in the art. Such methods include, but are not limited to, melt recrystallization, melt cooling, solvent recrystallization, desolvation, rapid evaporation, rapid cooling, slow cooling, vapor diffusion, and sublimation.

Techniques for characterizing polymorphs include, but are not limited to, differential scanning calorimetry (DSC), X-ray powder diffractometry (XRPD), single-crystal X-ray diffractometry, vibrational spectroscopy, e.g., IR and Raman spectroscopy, solid-state NMR, hot stage optical microscopy, scanning electron microscopy (SEM), electron crystallography and quantitative analysis, particle size analysis (PSA), surface area analysis, solubility studies, and dissolution studies.

To “characterize” a solid form of a compound, one may, for example, collect XRPD data on solid forms of the compound and compare the XRPD peaks of the forms. For example, when only three solid forms, e.g., Forms X and Y and Material N, are compared. The Form X pattern shows a peak at an angle where no peaks appear in the Form Y or Material N pattern, then that peak, for that compound, distinguishes Form X from Form Y and Material N and further acts to characterize Form X. The collection of peaks that distinguish, e.g., Form X from the other known forms, may be used to characterize Form X. Those of ordinary skill in the art will recognize that there are often multiple ways to characterize solid forms, including using the same analytical technique. Additional peaks could also be used, but are unnecessary, to characterize the form up to include an entire diffraction pattern. Although all the peaks within an entire XRPD pattern may be used to characterize such a form, a subset of that data may, and typically is, be used to characterize the form.

An XRPD pattern is an x-y graph with a diffraction angle (typically ° 20) on the x-axis and intensity on the y-axis. The peaks within this pattern may be used to characterize a crystalline solid form. As with any data measurement, there is variability in XRPD data. The data are often represented solely by the diffraction angle of the peaks rather than including the intensity of the peaks because peak intensity can be particularly sensitive to sample preparation (for example, particle size, moisture content, solvent content, and preferred orientation effects influence the sensitivity), so samples of the same material prepared under different conditions may yield slightly different patterns: this variability is usually greater than the variability in diffraction angles. Diffraction angle variability may also be sensitive to sample preparation. Other sources of variability come from instrument parameters and processing of the raw X-ray data: different X-ray instruments operate using different parameters. These may lead to slightly different XRPD patterns from the same solid form, and similarly different software packages process X-ray data differently. This also leads to variability. These and other sources of variability are known to those of ordinary skill in the pharmaceutical arts. Due to such sources of variability, it is usual to assign a variability of ±0.2° 2θ to diffraction angles in XRPD patterns.

As used herein, the term “about” is intended to qualify the numerical values it modifies, denoting such a value as a variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

As used herein, “administering” means to provide a compound or other therapy, remedy, or treatment such that an individual internalizes a compound.

As used herein, the term “disease” is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, “in need of treatment” and “in need thereof” when referring to treatment are used interchangeably to mean a judgment made by a caregiver (e.g., physician, nurse, nurse practitioner, etc. in the case of humans: veterinarian in the case of animals, including non-human mammals) that an individual or animal requires or will benefit from treatment. This judgment is made based on a variety of factors in the realm of a caregiver's expertise, but that includes the knowledge that the individual or animal is ill, or will become ill, as the result of a disease, condition, or disorder that is treatable by the compounds of the invention. Accordingly, the compounds of the invention can be used in a protective or preventive manner; or compounds of the invention can be used to alleviate, inhibit or ameliorate the disease, condition, or disorder.

As used here, “pharmaceutical composition” means a composition comprising at least one active ingredient, such as vamorolone or a polymorphic form thereof, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pure” means about 90-100%, preferably 95-100%, more preferably 98-100% (wt/wt) or 99-100% (wt/wt) pure compound; e.g., less than about 10%, less than about 5%, less than about 2% or less than about 1% impurity is present. Such impurities include, e.g., degradation products, oxidized products, epimers, solvents, and/or other undesirable impurities.

When ranges of values are disclosed, and the notation “from n1 . . . to n2” is used, where n1 and n2 are the numbers, then unless otherwise specified, this notation is intended to include the numbers themselves and the range between them. This range may be integral or continuous between and including the end values. Thus, by way of example, the range “from 2 to 6 carbons” is intended to include two, three, four, five, and six carbons since carbons come in integer units. Compare, by way of example, the range “from 1 to 3 μM (micromolar),” which is intended to include 1 μM, 3 M, and everything in between to any number of significant figures (e.g., 1.255 μM, 2.1 μM, 2.9999 μM, etc.).

As used herein, the term “room temperature” refers to a temperature of 68 to 86 F.

As used herein, the term “stable” refers to both chemical (shelf-life) and physical stability (suspension uniformity). Improved uniformity results in an improved product because less shaking of the suspension is required before dosing and allows the product to be stored longer (i.e., longer shelf-life) because the drug in the product will not settle and compact.

As used herein, “suspension” refers to a mixture of a solid in a liquid. In contrast, an “emulsion” refers to a mixture of two immiscible liquids.

As used herein, the term “therapeutically acceptable” refers to those compounds (or salts, prodrugs, tautomers, zwitterionic forms, etc.) suitable for use in contact with the tissues of patients without undue toxicity, irritation, and allergic response, are commensurate with a reasonable benefit/risk ratio and are effective for their intended use.

As used herein, the phrase “therapeutically effective” is intended to qualify the amount of active ingredients used in the treatment of a disease or disorder. This amount will achieve the goal of reducing or eliminating the disease or disorder.

As used herein, “treating,” “treatment,” and the like means ameliorating a disease to reduce or eliminate its cause, its progression, its severity, or one or more of its symptoms, or otherwise beneficially alter the disease in a subject.

As used herein, “prevention” means complete protection from disease, such as in the case of prevention of infection with a pathogen, or may involve prevention of disease progression, for example, from prediabetes to diabetes. For example, prevention of a disease may not mean complete foreclosure of any effect related to the disease at any level. Instead, it may mean preventing the symptoms of a disease to a clinically significant or detectable level. Prevention of diseases may also mean prevention of the progression of a disease to a later stage of the disease. Prevention may be preemptive: i.e., it may include prophylaxis of disease in a subject exposed to or at risk for the disease.

As used herein, stunting of growth means a negative change in height percentile for age for a human patient. Stunting of growth is measured against age-normalized population-based normative curves in children (for example, see FIG. 5 and other clinical growth charts, based on age and sex) and quantified as percentiles against the population means. Stunting of growth may also be referred to as having or showing growth deceleration (e.g., linear growth deceleration). In contrast, a human patient not having or showing stunting of growth may be described as maintaining growth velocity or trajectory.

Abbreviations used herein include:
DMD, Duchenne muscular dystrophy
CINRG, Cooperative International Neuromuscular Research Group;
· DNHS, Duchenne Natural History Study;
· SD, standard deviation;
· SE, standard error;
· SEM, standard error of the mean;
TTCLIMB, time to climb four stairs;
TTRW, time to run/walk 10 meters;
TTSTAND, time to stand from supine;
6MWT, 6-minute walk test;
CI, confidence interval;
BMI, body mass index;
LS, least squares;
NA, not available;
NR, not reported: and
NSAA, North Star Ambulatory Assessment.

As used herein, a “dose” means the measured quantity of an active agent to be taken at one time by a patient.

As used herein, a “dosage” is the prescribed administration of a specific amount, number, and frequency of doses over a specific period of time.

As used herein, “risk” means the probability or chance of adverse reaction, injury, or other undesirable outcome arising from a medical treatment. An “acceptable risk” means measuring the risk of harm, injury, or disease arising from a medical treatment that an individual or group will tolerate. Whether a risk is “acceptable” will depend upon the advantages that the individual or group perceives to be obtainable in return for taking the risk, whether they accept whatever scientific and other advice is offered about the magnitude of the risk, and numerous other factors, both political and social. An “acceptable risk” of an adverse reaction means that an individual or a group in society is willing to take or be subjected to the risk that the adverse reaction might occur since the adverse reaction is one whose probability of occurrence is small or whose consequences are so slight, or the benefits (perceived or real) of the active agent are so great. An “unacceptable risk” of an adverse reaction means that an individual or a group in society is unwilling to take or be subjected to the risk that the adverse reaction might occur upon weighing the probability of occurrence of the adverse reaction, the consequences of the adverse reaction, and the benefits (perceived or real) of the active agent. “At-risk” means in a state or condition marked by a high level of risk or susceptibility. A risk assessment identifies and characterizes the nature, frequency, and severity of the risks associated with using a product.

As used herein, “safety” means the incidence or severity of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

As used herein, “down-titration” or “dose de-escalation” of a compound refers to decrease the amount of a compound to achieve a therapeutic effect that occurs before administration of the compound is terminated. Down-titration can be achieved in one or more dose increments, which may be the same or different.

As used herein, “up-titration” or “dose escalation” of a compound refers to increasing the amount of a compound to achieve a therapeutic effect that occurs before dose-limiting intolerability for the patient. Up-titration can be achieved in one or more dose increments, which may be the same or different.

As used herein, “maximum recommended total daily dose” or “maximum recommended daily dosage” or “maximum total daily dose” or “maximum daily dosage” or “total daily dosage” refers to the highest safe dosage of a drug to be administered daily following dosage titration, i.e., the maintenance dose, as determined by a titration scheme, should not exceed the maximum recommended total daily dose.

Throughout this specification, unless the context requires otherwise, the word “comprise,” or variations such as “comprises” or “comprising” will be understood to imply the inclusion of a stated step or element or integer or group of steps or elements or integers but not the exclusion of any other step or element or integer or group of elements or integers.

Throughout this specification, unless expressly stated otherwise or the context requires otherwise, reference to a single step, composition of matter, group of steps, or group of compositions of matter shall be taken to encompass one and a plurality (i.e., one or more) of those steps, compositions of matter, groups of steps, or groups of compositions of matter.

Each embodiment described herein is to be applied mutatis mutandis to each other embodiment unless expressly stated otherwise.

Those skilled in the art will appreciate that the invention(s) described herein is susceptible to variations and modifications other than those specifically described. It is to be understood that the invention(s) includes all such variations and modifications. The invention(s) also includes all the steps, features, compositions, and compounds referred to or indicated in this specification, individually or collectively, and all combinations or any two or more steps or features unless expressly stated otherwise.

The present invention(s) is not limited in scope by the specific embodiments described herein, which are intended for exemplification only. Functionally equivalent products, compositions, and methods are clearly within the scope of the invention(s), as described herein.

It is appreciated that certain features of the invention(s), which are, for clarity, described in the context of separate embodiments, can also be provided in combination in a single embodiment. Conversely, various features of the invention(s), which are, for brevity, described in the context of a single embodiment, can also be provided separately or in any suitable subcombination.

Provided is a method of treating or reducing the symptoms of congenital adrenal hypoplasia in a human patient, comprising administering to the human patient in need thereof a therapeutically effective amount of a compound having the structural formula

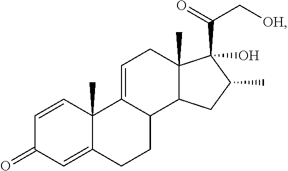 [SMILES: C[C@@H]1CC2C3CCC4=CC(=O)C=C[C@]4(C)C3=CC[C@]2(C)[C@@]1(O)C(=O)CO]

or a salt or polymorph thereof.

In certain embodiments, the symptoms of adrenal insufficiency are chosen from weakness, fatigue, loss of appetite, weight loss, vomiting, difficulty with feeding, dehydration, hypoglycemia, low sodium levels (hyponatremia), and shock.

In certain embodiments, the CAH is salt-wasting CAH. Salt-wasting CAH presents in the first weeks of life with failure to thrive, recurrent vomiting, dehydration, hypotension, hyponatremia, hyperkalemia, and shock.

In certain embodiments, the CAH is simple-virilizing adrenal hyperplasia (SVAH). SVAH is identified later in childhood because of precocious pubic hair, clitoromegaly, or both, often accompanied by accelerated growth and skeletal maturation due to excess postnatal exposure to adrenal androgens.

In certain embodiments, the CAH is non-classical adrenal hyperplasia. In certain embodiments, the CAH is characterized by a deficiency in 21-hydroxylase, 3ß-hydroxysteroid dehydrogenase, or 17-hydoxylase. Milder deficiencies of 21-hydroxylase or 3β-hydroxysteroid dehydrogenase activity may present in adolescence or adulthood with oligomenorrhea, hirsutism, and/or infertility. Females with 17-hydroxylase deficiency appear phenotypically female at birth but do not develop breasts or menstruate in adolescence because of inadequate estradiol production. They may present with hypertension.

In certain embodiments, the patient is male and the CAH is characterized by a 21-hydoxylase deficiency. 21-Hydroxylase deficiency in males is generally not identified in the neonatal period because the genitalia are normal. If the defect is severe and results in salt wasting, these male neonates present at age 1-4 weeks with failure to thrive, recurrent vomiting, dehydration, hypotension, hyponatremia, hyperkalemia, and shock. Patients with less severe deficiencies of 21-hydroxylase present later in childhood because of the early development of pubic hair, phallic enlargement, or both, accompanied by accelerated linear growth and advancement of skeletal maturation (simple-virilizing CAH).

In certain embodiments, the classical congenital adrenal hyperplasia is due to 21-hydroxylase deficiency.

In certain embodiments, the subject has a mutation in the CYP21A2 gene located on chromosome 6p21.

In certain embodiments, the subject does not have a mutation of the 11ß-hydroxylase gene CYP11B1 (11β-OH CAH).

In certain embodiments, prior to the administration, the subject exhibits an elevated 17-hydroxyprogesterone (17-OHP) level.

In certain embodiments, subsequent to the administration, the subject exhibits a reduced 17-OHP level as compared to the subject's 17-OHP level prior to administration.

In certain embodiments, prior to the administration, the subject exhibits an elevated adrenocorticotropic hormone (ACTH) level.

In certain embodiments, subsequent to the administration, the subject exhibits a reduced ACTH level as compared to the subject's ACTH level prior to administration.

In certain embodiments, prior to the administration, the subject exhibits an elevated androstenedione level.

In certain embodiments, subsequent to the administration, the subject exhibits a reduced androstenedione level as compared to the subject's androstenedione level prior to administration.

In certain embodiments, the patient was previously treated for CAH by normalizing hormone and steroid levels. These prior treatments can use various medications from diagnosis in infancy through adulthood. In certain embodiments, the prior treatments was one or more glucocorticoids.

In certain embodiments, the prior glucocorticoid treatment supported normal physiology and/or ensured sufficient cortisol for a strong stress response (e.g., intercurrent illness, exercise, and hypotension). In certain embodiments, careful monitoring avoided developing iatrogenic Cushing's syndrome due to glucocorticoid overtreatment to adequately suppress androgen production or Addisonian syndrome due to undertreatment. In certain embodiments, overtreatment with mineralocorticoids cause hypertension in the human patient. In certain embodiments, under-treatment with mineralocorticoids lead to low blood pressure, salt loss, fatigue, and increased glucocorticoid requirements. In certain embodiments, treatment efficacy is monitored using laboratory tests, including measuring plasma concentrations of 17-OHP, androstenedione, testosterone, renin activity, and electrolytes.

In certain embodiments, the method further comprises the administration of a mineralocorticoid replacement to achieve normal plasma renin activity to maintain regular blood pressure, electrolyte balance, and volume status in those patients with the salt-wasting form of CAH.

In certain embodiments, adult human patients with CAH have an increased prevalence of cardiovascular risk factors, including obesity, hypertension, and insulin resistance.

In certain embodiments, the human patient was treated with a therapeutically effective amount of glucocorticoid to normalize cortisol deficiency. In certain embodiments, the human patient is a child and the glucocorticoid is hydrocortisone. In certain embodiments, the human patient is an adult and the glucocorticoid is dexamethasone, which is more potent agent than hydrocortisone with a narrower therapeutic index. In certain embodiments, the human patient has the symptom of salt-wasting and was treated with mineralocorticoids, such as fludrocortisone.

In certain embodiments, the glucocorticoid doses for sufficiently suppressing excess androgens were above the normal physiologic dose for cortisol replacement alone, as in patients with Addison's disease. In certain embodiments, this increased exposure to glucocorticoids lead to the human patient developing one or more chosen from iatrogenic Cushing's syndrome, increased cardiovascular risk factors, glucose intolerance, and decreased bone mineral density.

In some embodiments, the treatment is characterized by fewer corticosteroid-associated safety concerns than a human patient treated with hydrocortisone, prednisone, dexamethasone.

In some embodiments, the corticosteroid-associated safety concern is chosen from bone fragility and fracture (e.g., spinal fracture), reduced or delayed growth (stunting of growth), hypogonadism, weight gain, behavioral effects (e.g., mood disturbance, irritability, or personality change), diabetes, hypertension, Cushingoid appearance, sleep disorder, hirsutism, and increased appetite.

In certain embodiments, the treatment is characterized by an increased velocity for time run/walk ten meters (TTRW). In certain embodiments, the TTRW velocity increased by at least 0.3 meters per second (e.g., 0.3 to 1 meter per second).

In certain embodiments, the treatment is characterized by an increased velocity for time to climb four stairs (TTCLIMB). In certain embodiments, the TTCLIMB velocity increased by at least 0.05 stairs per second (e.g., 0.05 to 1.5 stairs per second).

In certain embodiments, the patient is treated without decreasing the rate of growth in the human patient.

In some embodiments, growth is measured by a change in mean height percentile for age.

In some embodiments, the human patient has a positive growth trajectory.

In some embodiments, the human patient has an increase in height percentile of at least 6.

In certain embodiments, the patient is treated without increasing the incidence of vertebral fractures in the human patient.

In certain embodiments, the symptom may be an adverse event of special interest (AESI). In this context, AESIs are prespecified based on pre-defined MedDRA search criteria for eleven AESI categories for the corticosteroid class and then further stratified into AESI of at least moderate severity. The symptoms for treating DMD with corticosteroids include, but is not limited to, behavior adverse events, blood glucose related problems, gastrointestinal symptoms, increased arterial blood pressure, immune suppression/infections, skin/hair changes, cataracts/glaucoma, cushingoid features, weight gain, bone fractures, slow growth.

In certain embodiments, the behavior adverse event is chosen from abnormal behavior, aggression, agitation, anger, anxiety, emotional disorder, irritability, altered mood, mood swings, sleep disorder, initial insomnia, personality change, poor sleep quality, psychomotor hyperactivity, and skin laceration. In certain embodiments, the patient is treated without increasing the incidence of behavior adverse events in the human patient. In certain embodiments, the behavior adverse event is chosen from one or more of aggression, agitation, anger, emotional disorder, irritability, mood swings, sleep disorder, initial insomnia, and personality change. In certain embodiments, the behavior adverse event is chosen from one or more of anger, mood swings, and personality change.

In certain embodiments, the patient is assessed with a Pediatric Anxiety Rating Scale (PARS) III questionnaire. The PARS is a dimensional measure of treatment efficacy. The PARS is a clinician-rated measure of symptom severity and associated impairment that targets generalized anxiety disorder (GAD), social phobia (SoP), and separation anxiety disorder (SAD). The PARS consists of a checklist of 50 anxiety symptoms (encompassing SAD, SoP, and GAD) and seven global items administered to the child and parent together. Global items are each rated on a six-point (0-5) scale and reflect the number of symptoms present, their frequency, the severity of anxiety feelings, the severity of physical symptoms of anxiety, overall avoidance of anxiety-provoking situations, and anxiety-related interference with functioning at and outside of the home.

The PARS has acceptable psychometric properties and is sensitive to cognitive behavioral therapy (CBT) and pharmacological treatment changes. The comprehensiveness of the PARS is appealing in light of symptom overlap and high rates of comorbidity across anxiety disorders The PARS is time-efficient, taking approximately 20-30 minutes to complete. Thus, the PARS is feasible for routine clinical care like other interview-based rating scales for assessing severity and treatment response, such as the Children's Depression Rating Scale-Revised and the Children's Yale-Brown Obsessive Compulsive Scale (CY-BOCS).

“Treatment response” is an improvement of sufficient magnitude such that the individual is no longer fully symptomatic but may continue to evince more than minimal symptoms. Treatment response is often operationalized as a significant reduction in symptom severity and/or functional impairment. “Remission” is the absence or near absence of symptoms after treatment, such as treating childhood disorders impacted by residual symptoms during development. Relative to treatment response, remission is a more conservative standard. Remission has been operationalized using binary measures of diagnostic status or dichotomized ratings on dimensional measures of global functioning, which correspond to youth being “disorder free.” Both treatment response and remission are defined a priori and measured using multiple sources of information.

In certain embodiments, the patient is treated without decreasing lean body composition and bone density in the human patent. In certain embodiments, the body composition and bone density are measured via dual-energy X-ray absorptiometry (DXA). DXA measures bone mineral density (BMD) using spectral imaging. Two X-ray beams with different energy levels are aimed at the patient's bones. When soft tissue absorption is subtracted out, the BMD can be determined from the absorption of each beam by bone.

In certain embodiments, the human patient's body composition is leaner than in the human patient taking a therapeutically effective amount of hydrocortisone for congenital adrenal hypoplasia.

The recommended starting replacement dosage of hydrocortisone for pediatric patients is 8 to 10 mg/m2 daily for treating adrenal insufficiency. For a human patient with CAH who is growing, the recommended dose of hydrocortisone is 10-15 mg/m2 per day. Higher doses may be needed based on the patient's age and symptoms. Lower starting doses may be sufficient in patients with residual but decreased endogenous cortisol production. Generally, 20 mg of hydrocortisone is considered to be equivalent to 5 mg of prednisolone.

Recommended doses of corticosteroids for DMD are prednisone (0.75 mg/kg/day) and deflazacort (0.9 mg/kg/day). However, a study of 340 DMD boys showed both drugs to be underdosed to mitigate safety concerns, with the mean average dose for daily prednisone 0.56 mg/kg/day (75% of recommended), and daily Emflaza™ 0.75 mg/kg/d (83% of recommended) (Bello et al., “Prednisone/prednisolone and deflazacort regimens in the CINRG Duchenne Natural History Study.” Neurology. 2015 85(12): 1048-55). In the same study, Emflaza™ showed higher frequencies of growth delay, cushingoid appearance, and cataracts than prednisone. Id. Other approved treatments for DMD (viltolarsen, etiplersen, golodirsen, casimersen) are mutation-specific, targeting small subpopulations of DMD patients, and are used as an add-on to corticosteroids. These are not considered available therapies as they were granted accelerated approval based on a surrogate endpoint.

In certain embodiments, the human patient's bone density is greater than in the human patient taking a therapeutically effective amount of hydrocortisone for congenital adrenal hypoplasia.

In certain embodiments, the total body lean mass index of the human patient showed greater positive changes in the human patient who has taken a therapeutically effective amount of hydrocortisone for congenital adrenal hypoplasia. Lean body mass (LBM), sometimes conflated with fat-free mass, is a component of body composition. Fat-free mass (FFM) is calculated by subtracting body fat weight from total body weight; total body weight is lean plus fat. LBM can be measured by DXA and estimated mathematically, such as with the Boer or Hume formulas and other methods available to a person of skill in the art. The positive changes to LBM are quantified by comparing the LBM of the human patient treated with vamorolone to a similar human patient taking prednisone. In certain embodiments, the positive change in total body lean mass index is at least 1%, such as at least 5% or at least 10%.

In certain embodiments, the rate of osteoporosis in the human patient is less than in the human patient taking a therapeutically effective amount of hydrocortisone for congenital adrenal hypoplasia.

In certain embodiments, the difference between chronological age of the human patient and the bone age of the human patient is reduced. A child's bone age (also called the skeletal age) is assigned by determining which of the standard X-ray images in the atlas most closely match the appearance of the child's bones on the X-ray. A difference between a child's bone age and chronological age might indicate a growth problem. The larger the difference between the bone age of a human patient and their chronological age, the greater the growth problem or disease symptom. When this difference between the chronological age and bone age is reduced, the severity of the growth problem or disease symptom is also reduced.

In certain embodiments, wherein the human patient demonstrates reduced positive transcriptional activity. “Positive transcriptional activity” refers to binding a specific protein (activator) for transcription to begin. DNA-bound activators can regulate transcription by helping with ignition. To do this, they sometimes tether RNA polymerase to the promoter. When positive transcriptional activity is reduced, as, in the disclosed methods, the binding of the specific protein is showed or inhibited, thus slowing or delaying the start of transcription. In certain embodiments, the reduction in positive transitional activity is by at least 1%, such as at least 5% or at least 10%.

In certain embodiments, the human patient also has muscular dystrophy. In some embodiments, the muscular dystrophy is chosen from Duchenne muscular dystrophy, Becker muscular dystrophy, limb-girdle muscular dystrophy, congenital muscular dystrophy, facioscapulohumeral muscular dystrophy, myotonic muscular dystrophy, oculopharyngeal muscular dystrophy, distal muscular dystrophy, and Emery-Dreifuss muscular dystrophy. In certain embodiments, the muscular dystrophy is chosen from Duchenne muscular dystrophy and Becker muscular dystrophy. In certain embodiments, the muscular dystrophy is Duchenne muscular dystrophy. Adrenal insufficiency and DMD are common comorbidities. One reason is that the major adrenal insufficiency gene, DAX1, neighbors the DMD gene at Xp21 and sometimes shows up as contiguous gene deletion syndrome.

In some embodiments, the signs or symptoms of Duchenne muscular dystrophy comprise one or more of progressive proximal weakness with onset in the legs and pelvis, hyperlordosis with wide-based gait, hypertrophy of weak muscles, pseudohypertrophy (enlargement of calf and deltoid muscles with fat and fibrotic tissue), reduced muscle contractility on electrical stimulation in advanced stages of the disease, delayed motor milestones, progressive inability to ambulate, heel cord contractures, paralysis, fatigue, skeletal deformities including scoliosis, muscle fiber deformities, cardiomyopathy, congestive heart failure or arrhythmia, muscular atrophy, and respiratory disorders.

In certain embodiments, the method further comprises measuring the subject's 17-OHP level prior to administration. In certain embodiments, the measurement is conducted using an immunoassap, such as a lanthanide fluoroimmunoassay.

In certain embodiments, the method further comprises conducting a biochemical and/or molecular genetic screening test between 8 and 14 days of life, prior to administration. In certain embodiments, the biochemical method includes immunoassay with organic solvent extraction or liquid chromatography followed by tandem mass spectrometry to measure steroid ratios of 17-OHP, androstenedione, and 21-deoxycortisol to cortisol. The genetic screen looks for CYP21A2 mutations that are associated with CAH.

In certain embodiments, the administration is for at least 6 months, such as at least 12 months, at least 18 months, at least 24 months, or at least 30 months. In certain embodiments, the months are consecutive. In certain embodiments, the months are cumulative.

In certain embodiments, between about 1 mg/kg/day and about 12 mg/kg/day of the compound is administered, such as between about 2 mg/kg/day and about 6 mg/kg/day of the compound. In certain embodiments, about 2 mg/kg/day of the compound is administered. In certain embodiments, the administration of 2 mg/kg/day of the compound has a decreased risk of weight gain for the human patient. In certain embodiments, about 6 mg/kg/day of the compound is administered.

In some embodiments, the human patient is a child. In certain embodiments, the human patient is 1 day to 18 years old, such as between 2 and 18 years old, between 4 and 12 years old, or between 4 and 7 years old.

In some embodiments, congenital adrenal hypoplasia is typically diagnosed in young children but can be, and has been, diagnosed in utero by gene test and confirmatory fetal muscle biopsy. Accordingly, patients may be treated as soon after birth as a physician deems appropriate.

In certain embodiments, the human patient is male. In certain embodiments, the human patient is female.

In certain embodiments, the compound is administered orally. In certain embodiments, the compound is administered as a solution or suspension. In certain embodiments, the solution or suspension comprises about 4 wt. % of the compound. In certain embodiments, the solution or suspension further comprises a flavoring agent.

In some embodiments, the vamorolone, or a salt or polymorph thereof, is administered via a titration scheme. In some embodiments, the goal of the titration scheme is to achieve an optimal level of disease control in which the patient is tolerating the treatment regimen, or has achieved satisfactory treatment, or, in the case of up-titration, until the maximum permitted dose is reached, or, in the case of down-titration, until the administration of the vamorolone, or a salt or polymorph thereof, is terminated.

In some embodiments, the vamorolone, or a salt or polymorph thereof, is administered via a titration scheme that comprises the down-titration of the vamorolone, or a salt or polymorph thereof, until a maintenance dose is administered.

In some embodiments, the down-titration scheme comprises:
administering an initial dose of the vamorolone, or a salt or polymorph thereof,
monitoring the reduction of symptoms and tolerability of the patient to the treatment,
administering a reduced dose of the vamorolone or a salt or polymorph thereof.

In some embodiments, the cycle of monitoring and reducing the dose that is administered is repeated until a maintenance dose is administered.

In some embodiments, the initial dose in a down-titration scheme is about 6 mg/kg/day. In some embodiments, the initial dose is about 5 mg/kg/day. In some embodiments, the initial dose is about 4 mg/kg/day. In some embodiments, the initial dose is about 3 mg/kg/day.

In some embodiments, for each cycle of reduction, the dose is reduced by an increment of about 0.5, about 1.0, about 1.5, about 2.5, about 3, about 3.5, or about 4 mg/kg/day. In some embodiments, the increment is about 0.5 mg/kg/day. In some embodiments, the increment is about 1 mg/kg/day. In some embodiments, the increment is about 1.5 mg/kg/day. In some embodiments, the increment is about 2 mg/kg/day. In some embodiments, the increment is about 2.5 mg/kg/day. In some embodiments, the increment is about 3 mg/kg/day. In some embodiments, the increment is about 3.5 mg/kg/day. In some embodiments, the increment is about 4 mg/kg/day.

In some embodiments, the initial dose is about 6 mg/kg/day and the reduced dose is about 2 mg/kg/day.

In some embodiments, the vamorolone, or a salt or polymorph thereof, is administered via a titration scheme that comprises the up-titration of the vamorolone, or a salt or polymorph thereof, until a maintenance dose is administered.

In some embodiments, the up-titration scheme comprises:
administering an initial dose of the vamorolone, or a salt or polymorph thereof,
monitoring the reduction of symptoms and tolerability of the patient to the treatment,
administering an increased dose of the vamorolone, or a salt or polymorph thereof.

In some embodiments, the cycle of monitoring and increasing the dose that is administered is repeated until a maintenance dose is administered.

In some embodiments, the initial dose for the up-titration scheme is about 2 mg/kg/day. In some embodiments, the initial dose is about 2.5 mg/kg/day. In some embodiments, the initial dose is about 3 mg/kg/day. In some embodiments, the initial dose is about 3.5 mg/kg/day.

In some embodiments, for each cycle, the dose is increased by an increment of about 0.5, about 1, about 1.5, about 2, about 2.5, about 3, about 3.5, or about 4 mg/kg/day. In some embodiments, the increment is about 0.5 mg/kg/day. In some embodiments, the increment is about 1.0 mg/kg/day. In some embodiments, the increment is about 1.5 mg/kg/day. In some embodiments, the increment is about 2 mg/kg/day. In some embodiments, the increment is about 2.5 mg/kg/day. In some embodiments, the increment is about 3 mg/kg/day. In some embodiments, the increment is about 3.5 mg/kg/day. In some embodiments, the increment is about 4 mg/kg/day.

In some embodiments, the initial dose is about 2 mg/kg/day, and the increased dose is about 6 mg/kg/day.

In some embodiments, the maintenance dose is about 6 mg/kg/day. In some embodiments, the maintenance dose is about 5.5 mg/kg/day. In some embodiments, the maintenance dose is about 5 mg/kg/day. In some embodiments, the maintenance dose is about 4.5 mg/kg/day. In some embodiments, the maintenance dose is about 4 mg/kg/day. In some embodiments, the maintenance dose is about 3.5 mg/kg/day. In some embodiments, the maintenance dose is about 3 mg/kg/day. In some embodiments, the maintenance dose is about 2.5 mg/kg/day. In some embodiments, the maintenance dose is about 2 mg/kg/day. In some embodiments, the maintenance dose is about 1.5 mg/kg/day. In some embodiments, the maintenance dose is about 1 mg/kg/day.

### EXAMPLES

The following examples are included to demonstrate some embodiments of the disclosure. It should be appreciated by those of skill in the art that the techniques disclosed in the examples represent techniques discovered by the inventors to function well in the practice of the disclosure. Those of skill in the art should, however, in light of the present disclosure, appreciate that many changes can be made in the specific embodiments disclosed and still obtain a like or similar result without departing from the spirit and scope of the disclosure, therefore all matter set forth is to be interpreted as illustrative and not in a limiting sense.

### Example 1: Preparation of Vamorolone

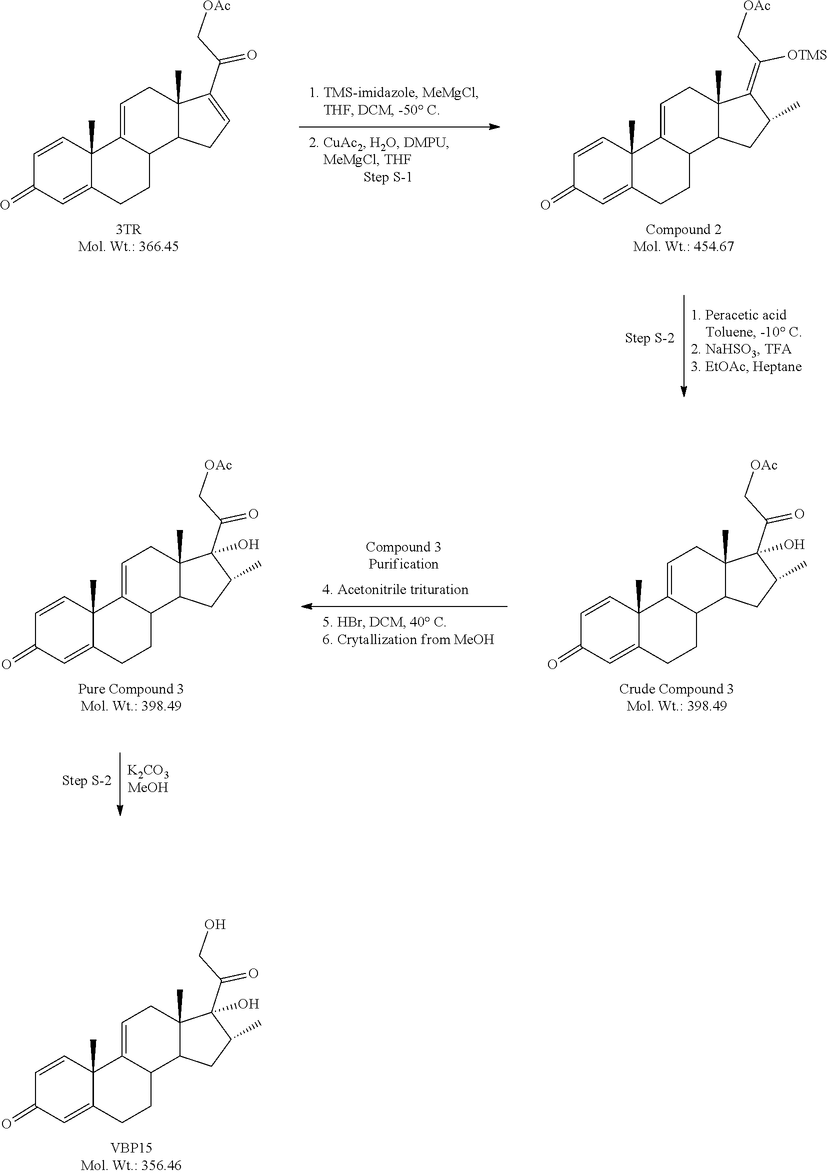

### Step 1—Compound 2 Preparation

2-((10S,13S)-10,13-dimethyl-3-oxo-6,7,8,10,12,13,14,15-octahydro-3H-cyclopenta[a]phenanthren-17-yl)-2-oxoethyl acetate (3-TR, 100 g, 273 mmol), dichloromethane (DCM, 500 mL), and tetrahydrofuran (THF, 400 mL) were charged to a reaction flask under nitrogen. To this was charged trimethylsilyl imidazole (TMS-imidazole, 65.3 g, 466 mmol, 1.7 eq). The resulting mixture was stirred at room temperature for 3 hours.

In a separate flask, copper acetate monohydrate (5.4 g, 27 mmol), tetrahydrofuran (400 ml), and 1,3-dimethyl-3,4,5,6-tetrahydro-2(1H)-pyrimidinone (DMPU, 53.3 g, 416 mmol) were combined and stirred at room temperature for approximately 3 hours. The blue mixture was subsequently cooled to −50° C., and to this was added methyl magnesium chloride solution (27 ml, 3.0 M in THF, 82 mmol) dropwise. After 30 minutes, the mixture had formed a deep blue, sticky “ball.”

The 3-TR/TMS-imidazole mixture was cooled to −50° C. and to this was charged the copper acetate/DMPU solution above via cannula. The residual sticky mass from the copper acetate/DMPU mixture was dissolved using DCM (50 mL) and transferred.

Methyl magnesium chloride (123.2 mL, 3.0 M solution in THF, 368 mmol) was added dropwise over 45 minutes to the combined reaction mixtures, which were then allowed to stir for 2 hours at −50° C. Subsequent HPLC analysis showed complete consumption of starting material. The mixture was allowed to warm to room temperature overnight, with stirring.

Toluene (800 mL) was added to the mixture, followed by a 5% acetic acid solution (600 mL). The aqueous layer was removed and discarded. The acetic acid wash was repeated. Next, the organic layer was washed with brine (400 mL), 5% sodium bicarbonate solution (400 mL×2), followed by a brine wash (400 mL). The organic solution was dried over sodium sulfate, then concentrated to dryness under reduced pressure. The product was recovered as a viscous, light golden oil. Mass recovery was 146 grams (119% theoretical).

### Step 2—Compound 3 Preparation

Compound 2 (92 g, 202 mmol) and toluene (1000 mL, 10.9 vol) were charged to a reaction flask under nitrogen, and the solution was cooled to −10° C. A 32 wt % solution of peracetic acid in acetic acid (60 mL, 283 mmol, 1.4 eq) was added dropwise over about 30 min maintaining the temperature at −10° C. The reaction was held for approximately 20 h (HPLC showed 75% Cmpd 3, Cmpd 2 1.5%, 6% diastereomer: 5% epoxide). Starting at −10° C., a 20% aqueous solution of sodium bisulfite (920 mL, 10 vol) was added carefully via an addition funnel, keeping the temperature below 10° C. Trifluoroacetic acid (16 mL, 202 mmol, 1 eq) was added, and the mixture was held for 3 h at 0-5° C. to complete desilylation (endpoint by HPLC). The lower aqueous layer was drained, and the organic layer was washed with a saturated solution of sodium bicarbonate (3×250 mL), followed by water (1×250 mL) and brine (1×150 mL). The organic layer was then dried over Na₂SO₄, filtered, and concentrated to a pasty solid (89 g). The residue was taken up in 1.5 vol of EtOAc and transferred to neat heptane (19 vol) to precipitate crude Cmpd 3 as an off-white solid (50 g, 62.5% yield; HPLC 79% Cmpd 3, 5.6% epoxide, 1.7% diastereomer). The crude Cmpd 3 (48.5 g) was triturated in hot acetonitrile (2 vol) at 60° C. for 4 h and then gradually cooled to ambient temperature overnight. The mixture was filtered using the recycled filtrate to rinse and wash the wet cake. After drying, the recovery was 64.3% (31.2 g; HPLC 93.5% Cmpd 3, 3.3% epoxide). To remove the epoxide impurity, the 31 Cmpd 3 was dissolved in DCM (250 mL, 8 vol), and a solution of 48% HBr in water was added (7.5 mL). The mixture was heated at 40 ° C. for 1 h (HPLC<0.3% epoxide). The mixture was cooled and transferred to a separatory funnel. The lower aqueous layer (brown) was removed, and the upper organic layer was washed with water (200 mL), saturated NaHCO₃ (150 mL), and brine (100 mL). The organic layer was dried over Na₂SO₄, filtered, and concentrated to a tan foam (32 g, ˜100% recovery). Methanol (64 mL, 2 vol) was added to the 32 g foam forming a slurry. To this was added a 1:1 solution of MeOH:water (60 mL, 2 vol) dropwise. The slurry cooled to slightly below ambient temperature and filtered using recycled filtrate to rinse and wash the wet cake. The solids were dried to constant weight, affording 26.1 g Cmpd 3 (81% recovery; HPLC 97.8%). The overall yield for Step 2 was 32.5%.

### Step 3—VBP15 Preparation

Compound 3 (26 g, 65 mmol) and MeOH (156 mL, 6 vol) were mixed in a reaction flask and cooled to 0-5° C. A solution of K2CO3 (9.9 g, 72 mmol, 1.1 eq) in water (65 mL) was added dropwise, and the mixture was allowed to gradually warm to ambient temperature overnight. Analysis by HPLC showed 2.5% SM and another 5 mol % K2CO3 was added, and the mixture stirred for another day (HPLC endpoint 1.1% Cmpd 3). The mixture was neutralized to pH 7 with 1.5 M HCl (53 mL), and ˜25% of the MeOH (30 g) was removed under vacuum to maximize recovery. After stirring for 2 days, the product was isolated by filtration using the recycled filtrate to transfer the wet cake to the funnel. The wet cake was dried under vacuum, affording 19.3 g VBP15 (83% yield) as an off-white powder. Analysis of the solids by HPLC showed 98.8% purity with 0.6% Cmpd 3 as the only major impurity.

### Example 2—Preparation of Aqueous Oral Pharmaceutical Suspension Compositions Comprising Vamorolone

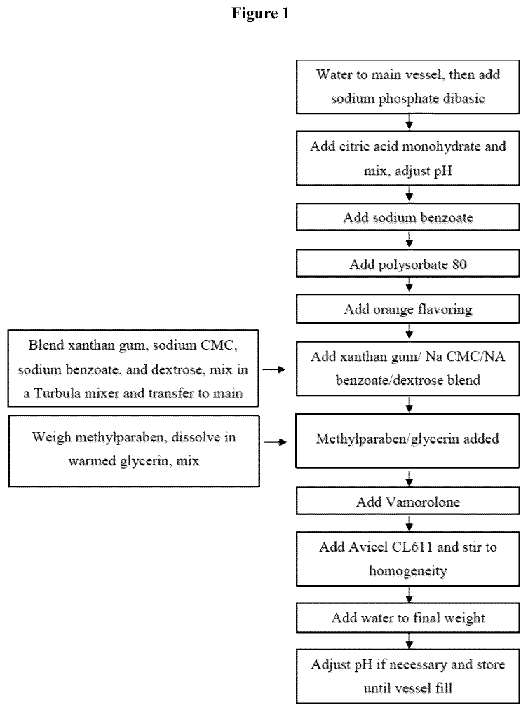

An oral pharmaceutical composition was prepared as a suspension by blending the ingredients in the amounts listed below in Table 1 to form a suspension. FIG. 1 shows a flow diagram for the manufacturing process used to prepare this suspension.

TABLE 1

| Ingredient | Amount (grams) |
|---|---|
| Vamorolone | 4.0 |
| Sodium Carboxymethylcellulose, Medium Viscosity, | 0.5 |
| USP | |
| Xanthan Gum, NF | 0.15 |
| Dextrose Anhydrous, USP | 1.0 |
| Polysorbate 80, NF | 0.1 |
| Avicel CL611 Microcrystalline cellulose, NF | 2.2 |
| Sodium Phosphate Dibasic, Anhydrous USP Grade | 0.19 |
| Citric Acid Monohydrate, Granular USP | 0.19 |
| Methylparaben, Ph. Eur./NF | 0.1 |
| Sodium Benzoate NF | 0.1 |
| Glycerin, USP | 5.0 |
| Orange flavor 58.4108.UL PHA | 0.1 |
| Sterile Purified Water, USP | Qs to 100 |

* “Qs” denotes the volume of sterile water necessary to bring the composition to 100 wt. %.

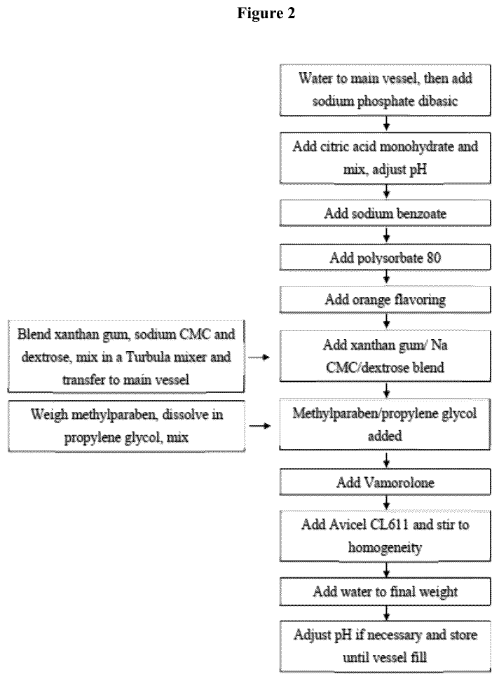

Another oral pharmaceutical composition was prepared as a suspension by blending the ingredients in the amounts listed below in Table 2 to form a suspension. FIG. 2 shows a flow diagram for the manufacturing process used to prepare this suspension.

TABLE 2

| Ingredient | Amount (grams) |
|---|---|
| Vamorolone | 4.0 |
| Sodium Carboxymethyl cellulose, Medium Viscosity, | 0.5 |
| USP | |
| Xanthan Gum, NF | 0.15 |
| Dextrose Anhydrous, USP | 1.0 |
| Polysorbate 80, NF | 0.1 |
| Avicel CL611 Microcrystalline cellulose, NF | 0.6 |
| Sodium Phosphate Dibasic, Anhydrous USP Grade | 0.19 |
| Citric Acid Monohydrate, Granular USP | 0.19 |
| Methylparaben, Ph. Eur./NF | 0.1 |
| Sodium Benzoate NF | 0.1 |
| Propylene Glycol, USP | 5.0 |
| Orange flavor 58.4108.UL PHA | 0.1 |
| Sterile Purified Water, USP | Qs to 100 |

* “Qs” denotes the volume of sterile water necessary to bring the composition to 100 wt. %.

Another oral pharmaceutical composition was prepared as a suspension by blending the ingredients in the amounts listed below in Table 3 to form a suspension.

TABLE 3

| Ingredient | Amount (grams) |
|---|---|
| Vamorolone | 4 |
| Xanthan Gum, NF | 0.3 |
| Dextrose Anhydrous, USP | 0.2 |
| Sodium Phosphate Dibasic, Anhydrous USP Grade | 0.28 |
| Citric Acid Monohydrate, Granular USP | 0.21 |
| Sodium Benzoate NF | 0.1 |
| Glycerin | 5 |
| Orange flavor 58.4108.UL PHA | 0.1 |
| Sterile Purified Water, USP | |

### Example 3: Phase 2 Clinical Trial in DMD

Vamorolone clinical studies have been conducted in adult male volunteers and boys with DMD, a disorder in which skeletal muscle is in a chronic inflammatory state. Two consecutive open-label dose-ranging studies in 48 DMD patients aged 4 to <7 years (corticosteroid-naïve) were conducted (Phase IIa, VBP15-002; Phase IIa, VBP15-003). Doses were tested over a 24-fold dose range (0.25, 0.75, 2.0, and 6.0 mg/kg/day), with 12 participants per group. The first multiple ascending dose (MAD) cohort trial-tested pharmacokinetics (PK) and safety for 2 weeks of drug dosing followed by a 2-week washout (VBP15-002). Vamorolone treatment showed no dose-limiting toxicities. PK demonstrated a short half-life similar to corticosteroids (˜2 hours), no drug accumulation, similar PK on day 1 and day 14 PK similar to that of healthy adult male volunteers (VBP15-001). All DMD participants completed the MAD study and then continued on the same dose for a 24-week dose-finding (efficacy and safety) extension study (VBP15-003). Oral administration of vamorolone at all doses tested was safe and well-tolerated over the 24-week treatment period. Participants in the 2 higher dose groups (2.0 and 6.0 mg/kg/day) generally showed clinical improvement of motor outcomes, suggesting dose-related improvements in all motor outcomes tested.

After completing the 24-week dose-finding study (VBP15-003), participants had the opportunity to enroll in a 24-month long-term extension study (VBP15-LTE) that permitted dose escalations and de-escalations. All trial participants' parents and physicians requested continued access to vamorolone rather than transition to the standard of care (prednisone or deflazacort). The initial experience from the 24-week VBP15-003 trial and the first 12 months of the 24-month VBP15-LTE trial (total 18 months of treatment) are reported below. In addition, changes in motor function and safety outcomes are compared to data from group-matched corticosteroid-treated and corticosteroid-naïve participants enrolled in the Cooperative International Neuromuscular Research Group (CINRG) Duchenne Natural History Study (DNHS). Safety endpoints (linear growth, body mass index) are also compared with data from a 12-month trial of daily prednisone (0.75 mg/kg group) in similar-aged boys with DMD.

### Methods

Three consecutive clinical trials of vamorolone treatment of DMD were conducted by CINRG (VBP15-002 [NCT02760264]: VBP15-003 [NCT02760277]: VBP15-LTE [NCT03038399]). A total of 48 participants (ages 4 to <7 years) were initially enrolled into VBP15-002, with trial participants completing month 12 of the 24-month VBP15-LTE study.

VBP15-002 (Phase IIa: two weeks on the drug, two weeks off drug) enrolled 48 corticosteroid-naïve participants with DMD, and all 48 participants completed the study and enrolled into VBP15-003 (Phase IIa extension: 24-week treatment). Forty-six of 48 participants completed the VBP15-003 study (2 participants withdrew from VBP15-003 for reasons unrelated to the study drug). In addition, all participants (46/46) opted to enroll in the 24-month long-term extension study, VBP15-LTE.

The consecutive vamorolone trials (VBP15-002, VBP15-003, VBP15-LTE) were open-label with no placebo comparator. Corticosteroid-naïve and corticosteroid-treated DMD participant comparators were group-matched participants from the CINRG DNHS (NCT00468832). The CINRG DNHS was an observational, prospective case-control study of 551 participants (440 with DMD, 111 healthy peers). For group matching between vamorolone-treated participants and CINRG DNHS participants, prespecified criteria were defined for matching within the interim statistical analysis plan (iSAP). Age-matched CINRG DNHS participants included those continuously corticosteroid-naïve over an 18-month period (n=19) or continuously corticosteroid-treated over an 18-month period (n=68). For the 68 corticosteroid-treated participants, as this was an observational cohort, corticosteroid doses and regimens varied based on clinician discretion. Although all 68 participants were treated for 18 months continuously, the age at initiation of corticosteroids varied. Thus, the total duration of corticosteroid treatment was longer than 18 months for most participants.

For comparisons of growth trajectories of vamorolone- and corticosteroid-treated participants, a third external comparator of a CINRG 12-month prednisone clinical trial was used (daily treated arm, 0.75 mg/kg/day). As with the CINRG DNHS comparators, group-matching criteria were prespecified in the iSAP, and 2 independent statisticians carried out the participant matching. The efficacy data from the CINRG 12-month prednisone trial were not compared to those of the vamorolone-treated participants. There was no corresponding 12-month assessment in vamorolone-treated participants. (Assessments of vamorolone-treated trial participants were 0, 3, 6, and 18 months).

### Measurements

Assessments of efficacy were motor outcomes (primary outcome: time to stand from supine [TTSTAND]: secondary outcomes: time to run/walk 10 meters [TTRW], time to climb four stairs [TTCLIMB], distance covered in 6-minute walk test [6MWT], and the North Star Ambulatory Assessment [NSAA]). 6MWT and NSAA were not assessed in most CINRG DNHS participants and were not compared to vamorolone-treated participants. According to standard operating procedures, clinical evaluators were trained to harmonize the CINRG vamorolone, CINRG DNHS, and CINRG prednisone studies. Reliability of these outcomes (percent coefficient of variation) has been reported for the VBP15-002/VBP15-003 studies. Assessments were done at baseline (VBP15-002 entry), 24 weeks (VBP15-003 last visit), and 18 months (VBP15-LTE midpoint assessment at 12 months).

Standing height and weight were assessed at each study visit. Height z-score, body mass index (BMI: kg/m²), and BMI z-score were calculated centrally. AE reporting was done per protocol in the vamorolone trials.

### Study Design

Only participants completing VBP15-002 and VBP15-003 were eligible to enroll in VBP15-LTE. Participants received vamorolone at 1 of 4 dose levels (0.25, 0.75, 2.0, or 6.0 mg/kg/day) and at the same dose level in both the 4-week VBP15-002 trial and the 24-week VBP15-003 trial. If participants, their families, and their physicians wished to continue vamorolone treatment upon exiting the VBP15-003 trial, they were offered participation in the 24-month long-term extension (VBP15-LTE). The last visit of the VBP15-003 trial was commensurate with the first visit of the VBP15-LTE trial. In all studies, study medication was provided as 4% flavored liquid suspension and was dosed according to body weight and given once daily in the morning with food.

Study visits took place quarterly, including assessment of clinical laboratory results, vital signs, and AEs. All AEs were coded using the Medical Dictionary for Regulatory Activities (MedDRA version 19.0) system for reporting (preferred term and system organ class). Clinical efficacy assessments were performed at baseline of the VBP15-002 study, at six months (end of VBP15-003 study), and the 12-month midpoint visit of the VBP15-LTE study.

The VBP15-LTE protocol permitted multiple-dose escalations to the highest dose (6.0 mg/kg/day) at the participant's family and physician's discretion and permitted de-escalations. Site investigators were permitted to escalate a participant's dose to a higher dose level during the VBP15-LTE (6.0 mg/kg/day) once the participant had been on their initial dose in VBP15-LTE for at least one month, the next higher dose was determined to be safe in the VBP15-002 Phase IIa Study, and no safety issues with that dose had emerged in the VBP15-003 Phase IIa study.

Vamorolone-treated participants were initially enrolled into VBP15-002 and VBP15-003 in 4 dose groups (0.25, 0.75, 2.0, and 6.0 mg/kg/day: groups A-D). Upon entering VBP15-LTE, vamorolone group A participants had 2 or 3 sequential dose escalations and were treated with 2.0 or 6.0 mg/kg/day for the last 3-9 months of the 18 months: group B participants had 1 or 2 dose escalations and were treated with 2.0 or 6.0 mg/kg/day for the last 9-11 months. Groups C and D were treated for 18 months at 2.0 or 6.0 mg/kg/day (S1 Fig).

The current study is the first to evaluate the longer-term tolerability, efficacy, and safety of vamorolone in DMD. The VBP15-003 dose-finding study suggested that vamorolone doses of 2.0 and 6.0 mg/kg/day showed better efficacy and similar safety profiles than lower doses. Given the variable timing of dose escalations, it was prespecified that initial analyses of drug-related efficacy and safety would be limited to those participants who had 18 months of treatment with 2.0 mg/kg/day vamorolone or more (dose group C+dose group D: n=23). Outcomes for these participants were compared to a group-matched cohort from the CINRG DNHS over 18 months (corticosteroid-naïve, n=19: corticosteroid-treated, n=68). Participants were matched for age and treatment period (+1 month), matching criteria were prespecified in the statistical analysis plan. Two independent statisticians carried out the matching.

Growth trajectories and BMI before/after drug treatment were compared between these CINRG DNHS groups over 18 months and were also compared to the cohort of CINRG prednisone clinical trial participants who were treated with daily prednisone for the 12-month treatment period of the trial (n=12). Participants in the corticosteroid-treated CINRG DNHS group were treated for at least 18 months, but the total duration, dose, and regimens varied.

### Statistical Analysis

An interim statistical analysis plan was written (VBP15-LTE iSAP) (S1 iSAP). The VBP15-LTE iSAP prespecified analyses of the VBP15-LTE midpoint (12-month) assessments and comparisons to external comparators (corticosteroid-treated and corticosteroid-naïve participants from CINRG DNHS). The VBP15-LTE iSAP included all month 12 assessments of the 24-month VBP15-LTE study. The software used was SAS.

The statistical analyses were carried out in 2 sequential steps. First, groups and comparisons in the VBP15-LTE iSAP were prespecified. This iSAP included only those vamorolone-treated participants who had been on 2.0 or 6.0 mg/kg/day for the full 18-month treatment period (dose groups C+D) to avoid the confounding variable of multiple-dose escalations in dose groups A and B (S1 Fig). The second analysis was conducted post hoc after completion of the VBP15-LTE iSAP analyses, with dose stratification based on initial dose group in VBP15-002 (0.25 [group A], 0.75 [group B], 2.0 [group C], and 6.0 [group D] mg/kg/day).

Statistical analyses were done on paired longitudinal outcome data using an analysis of covariance (ANCOVA) approach with change from baseline (VBP15-002) to month 18 (midpoint of VBP15-LTE). Baseline response and age were included as covariates. For the vamorolone-treated participants, age was calculated as (date of informed consent minus birthdate)/365.25. For the DNHS participants, age was calculated as (date of baseline visit used minus birthdate)/365.25. The baseline visit for a DNHS participant was the first visit. Thus, the participant met the comparison eligibility criteria for matching and had a non-missing response for at least one endpoint of interest. Timed function tests were analyzed as velocity scores to limit the impact of participants who could not perform the test (velocity=0). Velocity measures are variance-stabilizing transformations, suppressing extreme raw outliers from raw values in seconds; these help with distributional assumptions of the statistical models/tests used. Raw data (seconds) are also reported. Velocity scores for TTSTAND (event/second), TTRW (meters/second), and TTCLIMB (event/second) were inputted as 0 at the first response missing due to inability to perform the test. All other data were observed values only, without imputation. No adjustments for multiplicity on inferential statistics were specified in the iSAP.

For within-group analysis, longitudinal change from baseline to 18 months was analyzed using a paired t-test. A longitudinal analysis was not performed for efficacy for the participants in the corticosteroid-treated CINRG DNHS study, as there was no baseline (pre-corticosteroid) efficacy assessment.

### Results

Demographic and baseline characteristics of the vamorolone-treated and comparator groups are provided in Table 4.

TABLE 4
Demographic and baseline characteristics.

| | | CINRG DNHS | | CINRG DNHS | | CINRG | |
|---|---|---|---|---|---|---|---|
| | VBP15-LTE | corticosteroid- | | corticosteroid- | | prednisone | |
| | (group C + D) | naïve | | treated | | trial | |
| Characteristic | (n = 23) | (n = 19) | | (n = 68) | | (n = 12) | |
| Age (years) | | | | | | | |
| Mean | 5.20 | 5.03 | | 5.96 | | 5.70 | |
| SD | 0.90 | 0.55 | | 0.64 | | 0.66 | |
| Median | 4.97 | 4.94 | | 6.05 | | 5.65 | |
| Minimum | 4.01 | 4.02 | | 4.25 | | 4.80 | |
| Maximum | 6.72 | 5.90 | | 6.99 | | 6.87 | |
| Race, n (%) | | | | | | | |
| Native American | 0 | 0 | | 0 | | 0 | |
| Asian | 0 | 3 | (15.8) | 7 | (10.3) | 2 | (16.7) |
| Black | 0 | 0 | | 0 | | 0 | |
| White | 23 (100) | 15 | (78.9) | 56 | (82.4) | 8 | (66.7) |
| Unknown | 0 | 1 | (5.3) | 1 | (1.5) | 0 | |
| Other | 0 | 0 | | 4 | (5.9) | 2 | (16.7) |
| Ethnicity, n (%) | | | | | | | |
| Hispanic or | 3 (13.0) | 0 | | 5 | (7.4) | 1 | (8.3) |
| Latino | | | | | | | |
| Not Hispanic or | 20 (87.0) | 19 | (100) | 63 | (92.6) | 11 | (91.7) |

| | | | | |
|---|---|---|---|---|
| Latino | | | | |
| Weight (kg) | | | | |
| Mean | 19.5 | 18.3 | 20.6 | 20.0 |
| SD | 2.5 | 2.0 | 3.4 | 3.5 |
| Median | 19.4 | 18.2 | 20.4 | 19.5 |
| Minimum | 15.1 | 15.6 | 15.1 | 16.3 |
| Maximum | 24.0 | 22.3 | 30.3 | 24.8 |
| Height (cm) | | | | |
| Mean | 107.0 | 105.4 | 109.2 | 110.3 |
| SD | 6.8 | 5.1 | 5.7 | 6.8 |
| Median | 107.7 | 105.0 | 109.0 | 108.7 |
| Minimum | 95.4 | 97.4 | 96.5 | 102.5 |
| Maximum | 117.5 | 114.0 | 124.3 | 126.5 |
| Body mass index | | | | |
| (kg/m²) | | | | |
| Mean | 17.0 | 16.4 | 17.2 | 16.5 |
| SD | 0.9 | 0.9 | 1.9 | 1.9 |
| Median | 16.9 | 16.4 | 16.7 | 16.6 |
| Minimum | 15.3 | 14.6 | 14.8 | 13.7 |
| Maximum | 18.6 | 18.3 | 24.2 | 20.0 |

CINRG, Cooperative International Neuromuscular Research Group; DNHS, Duchenne Natural History Study.

Forty-eight DMD participants were enrolled into VBP15-002 and entered into 4 vamorolone treatment groups (dose group A, 0.25 mg/kg/day; dose group B, 0.75 mg/kg/day; dose group C, 2.0 mg/kg/day; dose group D, 6.0 mg/kg/day). All 48 participants completed the 4-week VBP15-002 trial, and 46 completed the 24-week VBP15-003 trial at the same doses. All 46 participants completing the 24-week VBP15-003 study then opted to enroll in the 24-month long-term extension study (VBP15-LTE). The current study is the first to evaluate the longer-term tolerability, efficacy, and safety of vamorolone in DMD. The VBP15-003 dose-finding study suggested that the two higher vamorolone doses showed greater efficacy than the two lower doses.

One participant discontinued the study one month before the 12-month assessment (S1 Fig: participant 233504). This participant's 11-month early exit visit data were counted as 12-month study data for this analysis, per the prespecified iSAP. All participants in the 0.25- and 0.75-mg/kg/day groups in VBP15-003 dose-escalated to either 2.0 or 6.0 mg/kg/day. The timing of dose escalations varied between participants. Two participants in the 0.75-mg/kg/day VBP15-003 dose group escalated to 6.0 mg/kg/day, then later de-escalated to 2.0 mg/kg/day due to weight gain within the 12-month interim period.

### Tolerability of Dose Escalation

Within the VBP15-LTE study, each participant could have his dose of vamorolone increased to a higher dose or decreased to a lower dose by the site investigator as necessitated clinically. Of the 11 participants in the 0.25-mg/kg/day dose group at entry in the VBP15-LTE, the vamorolone dose was increased to 2.0 mg/kg/day for 3 participants and 6.0 mg/kg/day for 8 participants before the 12-month interim assessment. The cumulative exposure to high-dose vamorolone (2.0 or 6.0 mg/kg/day) for those originally in the 0.25-mg/kg/day dose group ranged from 3 to 9 months (of the 18-month study period). Of the 12 participants in the 0.75-mg/kg/day dose group at entry in the VBP15-LTE, the vamorolone dose was increased to 2.0 mg/kg/day for 6 participants and to 6.0 mg/kg/day for 6 participants. The cumulative exposure to high-dose vamorolone for those originally in the 0.75-mg/kg/day dose group ranged from 9 to 11 months. Of the 12 participants in the 2.0-mg/kg/day dose group at entry in the VBP15-LTE, the dose remained at 2.0 mg/kg/day for 3 participants and was increased to 6.0 mg/kg/day for 9 participants. Two participants subsequently had their vamorolone dose decreased from 6.0 to 2.0 mg/kg/day due to weight gain. All eleven participants in the 6.0-mg/kg/day/day at entry in the VBP15-LTE remained at this dose throughout the study.

### Efficacy Evaluation of Vamorolone-Treated Versus Corticosteroid-Naïve Participants

Participants treated for 18 months with vamorolone (2.0 or 6.0 mg/kg/day) showed significant improvements in all measures of efficacy (Table 5). Paired t tests were significant for longitudinal improvements in all outcomes from baseline (TTSTAND velocity, p=0.012 [95% CI 0.010, 0.068 event/second]; TTRW velocity, p<0.001 [95% CI 0.220, 0.491 meters/second]; TTCLIMB velocity, p=0.005 [95% CI 0.034, 0.105 event/second]; 6MWT, p=0.001 [95% CI 31.14, 93.38 meters]; NSAA total score, p<0.001 [95% CI 2.702, 6.662 points]). Group-matched corticosteroid-naïve participants from CINRG DNHS showed no change or slight improvements over this same time frame for TTRW velocity, TTCLIMB velocity, and TTSTAND velocity (6MWT and NSAA outcomes were not available in CINRG DNHS). ANCOVA comparisons between vamorolone-treated and corticosteroid-naïve participants did not show significant differences for TTSTAND (least squares [LS] mean 0.042 [95% CI −0.007, 0.091], p=0.088), but showed significant differences favoring vamorolone for TTRW velocity (LS mean 0.286 [95% CI 0.104, 0.469], p=0.003) and TTCLIMB velocity (LS mean 0.059 [95% CI 0.007, 0.111], p=0.027).

TABLE 5
Analyses of efficacy and safety outcome measures over 18 months, with
comparison to corticosteroid-naïve DNHS participants.

| | | | | | |
|---|---|---|---|---|---|
| | | | | | LS mean |
| | | | | Change from | difference |
| | | | | baseline (SD) | (SE) (95% 2- |
| Outcome and | n at | | | (95% 2-sided | sided CI), |
| treatment | baseline/ | Baseline | 18-month | CI), paired t | ANCOVA p- |
| group | 18 months | value (SD) | value (SD)¹ | test p-value | value |

Efficacy
TTSTAND velocity (event/second)

| | | | | | |
|---|---|---|---|---|---|
| Vamorolone | 23/22 | 0.206 (0.07) | 0.241 (0.076) | 0.039 (0.066) | 0.042 (0.024) |
| | | | | (0.010, 0.068) | (−0.007, 0.091) |
| | | | | p = 0.012 | p = 0.088 |
| Corticosteroid- | 19/17 | 0.202 (0.055) | 0.205 (0.102) | −0.003 (0.083) | |
| naïve DNHS | | | | (−0.046, 0.039) | |
| | | | | p = 0.877 | |

TTRW velocity (meters/second)

| | | | | | |
|---|---|---|---|---|---|
| Vamorolone | 23/22 | 1.735 (0.331) | 2.061 (0.347) | 0.356 (0.306) | 0.286 (0.09) |
| | | | | (0.220, 0.491) | (0.104, 0.469) |
| | | | | p < 0.001 | p = 0.003 |
| Corticosteroid- | 19/18 | 1.619 (0.483) | 1.717 (0.46) | 0.093 (0.281) | |
| naïve DNHS | | | | (−0.047, 0.232) | |
| | | | | p = 0.179 | |

TTCLIMB velocity (event/second)

| | | | | | |
|---|---|---|---|---|---|
| Vamorolone | 23/22 | 0.266 (0.134) | 0.331 (0.127) | 0.07 (0.08) | 0.059 (0.026) |
| | | | | (0.034, 0.105) | (0.007, 0.111) |
| | | | | p = 0.001 | p = 0.027 |
| Corticosteroid- | 19/18 | 0.218 (0.098) | 0.242 (0.108) | 0.021 (0.089) | |
| naïve DNHS | | | | (−0.023, 0.065) | |
| | | | | p = 0.330 | |

6MWT meters walked (meters)

| | | | | | |
|---|---|---|---|---|---|
| Vamorolone | 20/19 | 343.2 (64.3) | 395.6 (69.7) | 62.2 (60.5) | NA |
| | | | | (31.14, 93.38) | |
| | | | | p = 0.001 | |

NSAA score (of 34)

| | | | | | |
|---|---|---|---|---|---|
| Vamorolone | 23/22 | 19.9 (4.9) | 24.3 (4.7) | 4.7 (4.5) | NA |
| | | | | (2.702, 6.662) | |
| | | | | p < 0.001 | |

Safety
Mean height percentile for age

| | | | | | |
|---|---|---|---|---|---|
| Vamorolone | 23/22 | 29.19 (24.66) | 35.24 (29.82) | 6.92 (9.68) | Versus |
| | | | | (2.622, 11.209) | vamorolone |
| | | | | p = 0.003 | |
| Corticosteroid- | 19/18 | 25.76 (21.37) | 27.16 (21.17) | 0.176 (11.72) | 6.72 (3.48) |
| naïve DNHS | | | | (−5.653, 6.004) | (−0.332, 13.78) |
| | | | | p = 0.950 | p = 0.061 |
| Corticosteroid- | 68/68 | 20.09 (22.58) | 14.46 (22.69) | −5.63 (14.89) | 15.86 (3.70) |
| treated DNHS | | | | (−9.231, −2.026) | (8.51, 23.22) |
| | | | | p = 0.003 | p < 0.001) |
| Prednisone | 12/12 | 29.89 (29.15) | 26.14 (24.21) | −3.76 (10.44) | 10.37 (3.86) |
| trial¹ | | | | (−10.387, 2.877) | (2.49, 18.25) |
| | | | | p = 0.238 | p = 0.012 |

Mean BMI z-score

| | | | | | |
|---|---|---|---|---|---|
| Vamorolone | 23/22 | 1.03 (0.56) | 1.46 (0.62) | 0.411 (0.615) | Versus |
| | | | | (0.138, 0.683) | vamorolone |
| | | | | p = 0.005 | |
| Corticosteroid- | 19/18 | 0.70 (0.58) | 0.36 (0.77) | −0.345 (0.655) | 0.899 (0.204) |
| naïve DNHS | | | | (−0.671, −0.019) | (0.486, 1.31) |
| | | | | p = 0.039 | p < 0.001 |
| Corticosteroid- | 68/67 | 0.98 (0.85)* | 1.13 (0.92) | 0.145 (0.518) | 0.282 (0.146) |
| treated DNHS | | | | (0.019, 0.272) | (−0.01, 0.573) |
| | | | | p = 0.025 | p = 0.058 |
| Prednisone | 12/12 | 0.61 (1.27) | 1.068 (1.05) | 0.459 (0.407) | 0.066 (0.193) |
| trial¹ | | | | (0.200, 0.718) | (−0.328, 0.461) |
| | | | | p = 0.002 | p = 0.733 |

¹The 18-month value reflects the outcome at 12 months of treatment, as this was the duration for the prednisone trial.
*Baseline indicates mean BMI at the beginning of the 18-month continuous treatment with corticosteroids. Participants may have been initiated on corticosteroids before this visit.

Results from analysis of measures in seconds units showed significance for 18-month improvements in vamorolone-treated participants for TTRW (p<0.001 [95% CI −1.53, −0.59 seconds]), but not TTSTAND (p=0.48 [95% CI −1.90, 0.93 seconds]) or TTCLIMB (p=0.62 [95% CI −2.67, 1.62 seconds]) due to severe outliers increasing variance. ANCOVA comparisons between vamorolone-treated and corticosteroid-naïve participants showed a significant difference favoring vamorolone for TTRW (LS mean −0.84 [95% CI −1.54, −0.14 seconds], p=0.02), but not for TTSTAND (LS mean −1.15 [95% CI −2.87, 0.57 seconds], p=0.18) or TTCLIMB (LS mean −0.34 [95% CI −3.28, 2.59 seconds], p=0.81).

Participant-level data were analyzed graphically for the four vamorolone-treated groups relative to DNHS corticosteroid-naïve participants (FIGS. 3 and 4). Groups B, C, and D each showed improvements from baseline after 18 months of treatment compared to corticosteroid-naïve participants from CINRG DNHS. In contrast, group A outcomes were similar to those of corticosteroid-naïve participants. Of note, group A was treated for only 3 to 9 months with high-dose vamorolone and was also had a mean age 0.4 years older than that of the other groups at study entry (Group A, 5.2±1.0 years: Groups B, C, and D, 4.8±0.8 years). A cross-sectional comparison was carried out at 5.5-8.5 years of age (end of 18-month treatment period) (FIG. 4), with visualization of the mean baseline of each of the four vamorolone groups and the DNHS corticosteroid-naïve (n=19) and DNHS corticosteroid-treated comparators (n=68). Vamorolone dose groups B, C, and D showed motor function outcomes similar to those of corticosteroid-treated DNHS participants. Corticosteroid-naïve participants showed poorer performance, as did vamorolone group A. These data suggest that the benefit of vamorolone at 2.0 or 6.0 mg/kg/day may be similar in magnitude to that of corticosteroid at 18 months of treatment.

### Comparative Efficacy of Vamorolone Dose Groups

FIG. 3 shows the participant-level change from baseline after an 18-month treatment period. Vamorolone group A was treated with 2.0 or 6.0 mg/kg/day for the last 3-9 months of the 18 months, group B was treated with 2.0 or 6.0 mg/kg/day for the last 9-11 months, and groups C and D with 2.0 or 6.0 mg/kg/day for all 18 months. The specific dose of each participant at the end of the 18-month period is indicated (red=2.0 mg/kg/day: blue=6.0 mg/kg/day). Dose groups B, C, and D show mean improvements over baseline compared to matched corticosteroid-naïve participants from CINRG DNHS (n=19). FIG. 4 shows mean group cross-sectional analysis at age 5.5-8.5 years. The baseline mean is shown for each vamorolone-treated group (black line). The corticosteroid-treated natural history group (n=68) has no baseline shown, as the age at initiation of corticosteroids was variable. This panel shows improvement over baseline in vamorolone-treated groups B, C, and D. The cross-sectional data suggest an effect size similar to that of age-group-matched corticosteroid-treated participants in CINRG DNHS.

### Prespecified Safety Evaluation

Two measures of corticosteroid-associated safety concerns were prespecified in the iSAP: growth deceleration (stunting of growth measured by the change in mean height percentile for age) and body mass index (BMI) z-score.

### Growth Stunting

At baseline, the three groups (vamorolone, DNHS corticosteroid-treated, and DNHS corticosteroid-naïve) were generally short for age (mean 20th-29th height percentile for age). In addition, DNHS corticosteroid-naïve participants showed no change in growth trajectories over the 18 months. In contrast, corticosteroid-treated participants showed the expected deceleration of growth seen with chronic treatment with corticosteroids (−5.63 mean change in height percentile) (Tables 6 and 7 ♦ below).

TABLE 6
Mean change in height percentiles for age in DMD
children treated with deflazacort or vamorolone.

| | | LS Mean change in | | P-value |
|---|---|---|---|---|
| | | height percentiles | | deflazacort vs. |
| | N | for age | 95% CI | vamorolone* |
| Deflazacort | 40 | −11.4 | −15.5, −7.4 | 4.04 × 10⁻⁸ |
| Vamorolone | 22 | +6.9 | +2.6, +11.2 | |

*two-sample t-test with Welch correction

Vamorolone-treated participants showed a positive growth trajectory (+6.92 mean change in height percentile); this was not significantly different from the trajectory of the DNHS corticosteroid-naïve participants. However, comparing the growth velocities of vamorolone-treated to DNHS corticosteroid-treated participants over the 18 months showed a significant difference (LS mean 15.86 [95% CI 8.51, 23.22], p<0.001). There was also a significant difference when comparing vamorolone 18-month treatment to prednisone trial 12-month treatment (LS mean 10.37 [95% CI 2.49, 18.25], p=0.012). This suggests that vamorolone treatment does not stunt growth, whereas corticosteroid-related growth stunting is a well-recognized safety concern.

### Body Mass Index Z-Score

For the BMI z-score, the vamorolone-treated group had a normal mean BMI at baseline (z-score=1.03). In contrast, DNHS corticosteroid-naïve and CINRG prednisone trial participants had a lower mean BMI at baseline (z-score=0.70 and 0.61, respectively). CINRG corticosteroid-naïve participants showed a decrease in mean BMI over 18 months (change of z-score=−0.34). Participants in the CINRG prednisone clinical trial showed an increase of mean z-score of 0.46 over 12 months of treatment, and the vamorolone group showed an increase of mean z-score of 0.41 over 18 months of treatment. CINRG DNHS participants treated with corticosteroids over 18 months showed an increase of mean z-score of 0.15, but this group did not have measures before initiation of corticosteroids. The change in BMI was not significantly different between vamorolone- and corticosteroid-treated groups, whereas comparisons of drug-treated groups to corticosteroid-naïve participants showed significant differences (Table 4, FIG. 6). Stratification by original dose groups shows a general dose-response of increasing BMI (change from baseline to 18 months of treatment [kg/m²]: group B, 0.5: group C, 1.11: group D, 2.55) with increasing vamorolone dose, although this was highly variable within all groups. This suggests that patients may gain weight when taking vamorolone like patients who take corticosteroids.

Comparison of vamorolone- to corticosteroid-treated DNHS participants showed a significant difference (p=8.94×10⁻⁰⁷) in mean change in height percentile, with no evidence of growth stunting in the vamorolone treatment group (FIG. 7). In addition, 39/41 LTE participants had wrist x-ray at Month 24; the mean bone age to chronologic age difference was −1.1 (p<0.001, CI −1.5, −0.7), indicating possible skeletal maturation delay (Table 7).

TABLE 7
Other Vamorolone* Pre-specified Study Outcomes

| | | | Change from |
|---|---|---|---|
| | | | baseline (SD) |
| Health-related | | 24 months from | Paired t-test p- |
| Quality of Life | LTE baseline | LTE baseline | value (n) |
| PODCI upper | 75.3 (15.1), n = 18 | 82.3 (10.9), n = 18 | +7.9 (12.5), |
| extremity and | | | p = 0.0278 (15) |
| physical function | | | |
| PODCI transfer and | 86.5 (9.2), n = 19 | 81.4 (17.5), n = 18 | −4.4 (22.7), |
| basic mobility | | | p = 0.451 (16) |

| Skeletal | LTE Month 24 | | Bone Age difference |
|---|---|---|---|
| Maturation by | Chronologic Age | LTE Month 24 | to Chronologic Age |
| wrist x-ray | (yr) | Bone Age (yr) | (yr) |
| Mean (SD) | 8.0 (1.0), n = 39 | 6.8 (1.4), n = 39 | −1.1 (1.2), |
| | | | p < 0.001 (39) |
| | | | (CI −1.5, −0.7) |

*Vamorolone assigned to high dose (2.0 and 6.0 mg/kg/day) at start of the study

PODCI is the Pediatrics Outcomes Data Collection Instrument (musculoskeletal disorders). The PODCI is a disease-specific questionnaire developed by the American Academy of Orthopedic Surgeons to measure general health and problems related to bone and muscle conditions in children. A significant increase in mean PODCI upper extremity and physical function standardized score was observed from LTE baseline to Month 24 (7.95±12.54, CI 1.003, 14.897, p=0.0278, n=15) for the combined 2.0 and 6.0 mg/kg/day initial dose group. At the same time, a non-significant decrease in mean PODCI transfer and basic mobility standardized score was observed (−4.38±22.68, CI −16.471, 7.703, p=0.451, n=16) (Table 7).

### Physician-Reported AEs

Treatment-emergent AEs (TEAEs) have been published for the 2-week treatment MAD study (VBP15-002) and the 24-week dose-finding extension study (VBP15-003) [14]. TEAEs were reported with similar incidence by participants in all four vamorolone groups. Several TEAEs commonly observed with chronic corticosteroid therapy were observed only in the 2.0-mg/kg/day group (abnormal behavior; one participant) and 6.0-mg/kg/day group (hypertrichosis [two participants] and anxiety, abnormal blood cortisol level, Cushingoid habitus, and personality change [one participant each]). The other reported TEAEs did not exhibit a dose-related incidence.

A Data and Safety Monitoring Board report on the VBP15-LTE study covering all participants enrolled in the VBP15-LTE study (inclusive beyond the 12-month midpoint assessment) included three serious AEs (2 myoglobinuria events [in the same participant] and one pneumonia), all deemed unrelated to study drug. For all reported TEAEs, 402 were deemed unrelated to vamorolone, 37 were deemed remotely related, 29 were deemed possibly related, 11 were deemed probably related, and three were deemed definitely related. Of the 14 AEs probably and definitely related to vamorolone, 10 were weight gain, 2 were increased appetite, 1 was Cushingoid features, and 1 was irritability.

The incidences of physician-reported AEs typically for corticosteroid treatment were determined for participants that had been treated with vamorolone 6.0 mg/kg/day (for any duration), taken from the March 2019 Data Safety Update Report (DSUR) (pharmacovigilance report). Incidence rates of Cushingoid features, weight gain, hirsutism, and behavior change were studied (Table 8). These rates were compared to physician-reported AE incidences in the CINRG DNHS study and a 12-month clinical trial of prednisone and deflazacort. This comparison showed lower rates of physician-reported Cushingoid features, weight gain, hirsutism, and behavior change in the vamorolone trial than published prednisone and deflazacort trials in boys with DMD.

TABLE 8
Incidence of physician-reported adverse events.

| | | n; mean | | | | |
|---|---|---|---|---|---|---|
| | | age in | | | Hyper- | |
| | | years | | Weight | trichosis/ | Behavior |
| Study | Treatment | (SD)¹ | Cushingoid | gain | hirsutism | change |
| Vamorolone | 6.0 | n = 38; | 2.6% | 13.2% | 0% | 0%² |
| | mg/kg/day | 4.9 | | | | |
| | vamorolone | (0.9) | | | | |
| Griggs et al. | 0.9 | n = 68; | 60.3% | 27.9% | 35% | 9% |
| | mg/kg/day | 8.8 | | | | |
| | deflazacort | (2.5) | | | | |
| 2016 | 0.75 | n = 63; | 77.8% | 34.9% | 44% | 14% |
| | mg/kg/day | 8.9 | | | | |
| | prednisone | (2.9) | | | | |
| CINRG | Deflazacort | n = 94 | 72% | 63% | NR | 33% |
| DNHS | Prednisone | n = 80 | 50% | 67% | NR | 30% |

Vamorolone data are from Data Safety Update Report 13 Mar. 2019 (data cutoff 9 Jan. 2019). Data shown are physician-reported adverse events.
¹Mean age shown for vamorolone is a cross-sectional analysis; the mean age shown for Griggs et al. is at baseline.
²No behavior change was reported, but one personality change, one sleep disorder, and two irritability were reported.

### Adrenocorticotropic Hormone (ACTH) Measurements

Two tests for adrenal insufficiency were used: morning cortisol (before drug) and ACTH stimulation tests. Adrenal suppression was measured via morning serum cortisol levels at Baseline, 12-weeks, and 24-weeks. The ACTH stimulation test was measured at Baseline and 24-weeks. Morning cortisol was measured in the clinic before daily drug administration. Morning cortisol levels were similar at Baseline (˜200 nmol/L) in all groups and remained stable over the 24-week trial in the Placebo Group. All other treatment groups showed significant reductions of morning cortisol at both 12-week and 24-week assessments by within-group paired T-tests. Vamorolone 2.0 mg/kg/day group showed less adrenal suppression than prednisone (p=0.002). Vamorolone 6.0 mg/kg/day was not significantly different from prednisone.

For ACTH stimulation tests, adrenal suppression was defined as the subject having stimulated cortisol levels<500 nmol/L (<18 μg/dL) at both 30- and 60-minutes following stimulation. At baseline, 9% (10/112) of participants showed <500 nmol/L at both time points. At Week 24, 20% of the placebo group, 100% of the prednisone group, 86% of the vamorolone 2.0 mg/kg/day group, and 95% of the vamorolone 6.0 mg/kg/day group had cortisol levels<500 nmol/L at both 30′ and 60′ after ACTH challenge, indicative of adrenal suppression. Fisher's exact test showed no significant differences between the vamorolone and prednisone groups for either time point.

The three active-treated groups showed significant reductions of morning cortisol at both 12-week and 24-week assessments. Still, the reductions observed with vamorolone 2.0 mg/kg/day were significantly less than those observed with prednisone. In addition, results of the ACTH stimulation tests, using a pre-specified threshold of <500 nmol/L cortisol level at 30 and 60 minutes as reflecting adrenal suppression, showed that about 10% of participants fell below this threshold at baseline 20% of the placebo group at 24-weeks.

The three active treatment groups showed a proportional increase of participants falling below this threshold at Week 24. There were no significant differences between drug treatment groups (the pre-specified analysis). While not pre-specified, an alternative analysis derived from meta-analysis, using basal cortisol as a screening tool, showed that DMD boys in the placebo group showed baseline adrenal insufficiency (25% of tests [n=12]<138 nmol/L [>92% chance of adrenal insufficiency]: 71% of tests [n=34] 138-365 nmol/L [40% chance of adrenal insufficiency]: 4% of tests [n=2]>365 nmol/L [<9% chance of adrenal insufficiency]). Part of the efficacy of corticosteroids and vamorolone may be in treating an underlying adrenal insufficiency.

### Discussion

A dose-ranging 24-week (6-month) study of vamorolone treatment in 4- to 7-year-old boys with DMD had shown dose-responsive improvements in motor function tests. After completing this study, participants were offered enrollment in a 2-year long-term extension study (VBP15-LTE) or transition to corticosteroid standard of care (deflazacort or prednisone). Interim findings in VBP15-LTE (18 months of vamorolone treatment) are reported herein. All participants (46 of 46) opted to enroll in the vamorolone long-term extension, suggesting high satisfaction with vamorolone treatment. Vamorolone-treated participants showed improvements from baseline in all five motor assessments over the 18-month treatment period (TTSTAND, TTRW, TTCLIMB, NSAA, and 6MWT) (Table 5). In contrast, group-matched steroid-naïve (non-treated) DMD participants in the CINRG DNHS study showed stable disease over a similar 18-month period. Comparing vamorolone-treated participants to CINRG DNHS non-treated participants showed that differences for TTSTAND were not significant, but significant vamorolone-related improvements were observed for TTRW velocity (p=0.003) and TTCLIMB velocity (p=0.027); data for NSAA and 6WMT were not available in the CINRG DNHS comparator group.

Vamorolone has shown fewer morbidities than corticosteroids in mouse disease models, but a comparative safety profile for vamorolone versus corticosteroids has not been previously reported in humans. Group-matched steroid-treated participants in the CINRG DNHS showed marked stunting of growth—a well-known safety concern with chronic deflazacort and prednisone treatment of children. In contrast, vamorolone-treated participants did not show any evidence of stunting of growth. In addition, physicians reported fewer other corticosteroid-associated safety concerns in vamorolone-treated participants than published studies of deflazacort- and prednisone-treated DMD patients, including Cushingoid appearance, behavior change (mood disturbance), hirsutism, and weight gain.

While participating in the VBP15-LTE study, participants were permitted dose escalations and de-escalations at the discretion of families and their physicians. Most (74%: 34/46) opted to be treated with the highest dose permitted (6.0 mg/kg/day), and 26% with 2.0 mg/kg/day. There were two participants for whom the vamorolone dose was decreased from 6.0 to 2.0 mg/kg/day due to weight gain. DMD trial participants treated with 2.0 or 6.0 mg/kg/day vamorolone for the full 18-month period (n=23) showed clinical improvement of all motor outcomes from baseline to month 18 (TTSTAND, p=0.012: TTRW, p<0.001: TTCLIMB, p=0.001; 6MWT, p=0.001: NSAA, p<0.001). However, DMD patients at this young age range are, on average, stable or improving. To interpret these results, clinical improvements should be compared to the control of non-treated participants.

The vamorolone clinical trials were conducted by the academic clinical trial network CINRG. The CINRG network had previously conducted a longitudinal natural history study of 551 DMD participants and healthy peers (CINRG DNHS), with similar clinical evaluator methods and endpoints used in the vamorolone trials. Prespecified matching criteria were defined to provide group matching of corticosteroid-naïve and corticosteroid-treated cohorts selected from the CINRG DNHS to compare to vamorolone-treated participants over 18 months. The comparator groups were similar to the vamorolone-treated groups at baseline, with slightly older ages in the CINRG DNHS study groups. These comparisons showed that DMD participants treated with vamorolone for 18 months (2.0 or 6.0 mg/kg/day) compared to corticosteroid-naïve participants did not show significant differences for TTSTAND velocity (p=0.088), but did show significant improvement for TTRW velocity (p=0.003) and TTCLIMB velocity (p=0.027) (Table 5). Vamorolone treatment led to improvements in the 6MWT (mean +62.2 meters) and NSAA (mean +4.7 points). Still, these outcomes were not measured over an 18-month interval in the CINRG DNHS, and, therefore, there was no group match comparator for these outcomes.

The cross-sectional graphical comparison of motor outcomes at the end of the 18-month treatment period (participants 5.5-8.5 years of age) is shown in FIG. 1. These outcomes suggest both the vamorolone-treated cohort (1 year or more treatment at 2.0 or 6.0 mg/kg/day: groups B, C, and D) and the CINRG DNHS corticosteroid-treated cohort had similar drug-related benefits relative to the CINRG DNHS corticosteroid-naïve cohort. Insufficient data were available to compare motor improvements with vamorolone to those natural history motor outcomes seen with specific corticosteroid regimens (e.g., daily prednisone and daily deflazacort).

Long-term treatment with corticosteroids (deflazacort and prednisone) is for a broad range of safety concerns that detract from the patient's quality of life. In children, deceleration of linear growth is frequently seen with chronic corticosteroid treatment. Comparison of mean height percentile change over 18 months showed that corticosteroid treatment in CINRG DNHS participants led to growth stunting (−5.63 percentile). Vamorolone treatment did not (+6.92 percentile) (p<0.001) (Table 5).

A double-blind clinical trial of prednisone versus deflazacort in DMD also found marked stunting of growth over a 12-month treatment period (−11.43 percentile for deflazacort: −7.04 percentile for prednisone) in all subjects. See Griggs R C et al., “Efficacy and safety of deflazacort vs. prednisone and placebo for Duchenne muscular dystrophy,” Neurology 87:2123-31 (2016). Consistent with the adverse effects of Emflaza on growth, the Label for Emflaza states: “5.10 Effects on Growth and Development. Long-term use of corticosteroids, including EMFLAZA, can have negative effects on growth and development in children.” In contrast, 18-months vamorolone treatment led to increases in height percentile for age in all subjects.

These data suggest that vamorolone does not share stunting of growth with corticosteroids as a safety concern, and this may be a distinct advantage for children requiring chronic corticosteroid treatment.

The DMD subjects' age ranges and treatment duration for the two studies differed (vamorolone 4 to 8.5 years, 18 months treatment: Emflaza 5 to 16 years, 12 months treatment). However, change over time for age-adjusted height percentiles are an objective outcome measure for child growth linear over the age ranges of DMD children in both studies.

The physician-reported incidence of adverse events was compared between the vamorolone trials, the corticosteroid-treated group in the CINRG DNHS, and the prednisone versus deflazacort trial (Table 6). This comparison suggested a lower incidence of Cushingoid appearance, weight gain, hirsutism/hypertrichosis, and behavior change in vamorolone-treated DMD patients compared to corticosteroid-treated boys. Taken together, the data suggest that vamorolone treatment of DMD patients provides similar efficacy as corticosteroid treatment as assessed by motor function outcomes. Furthermore, this preliminary assessment indicates that vamorolone treatment resulted in fewer safety concerns typical for corticosteroid treatment. The BMI data from the 18-month extension in comparison to natural history data from the CINRG do not indicate that vamorolone-treated participants will be wholly spared the side effects of weight gain, and two participants on 6.0 mg/kg/day had to de-escalate their dose to 2.0 mg/kg/day.

In conclusion, for boys with DMD, vamorolone treatment for 18 months is efficacious compared to a natural history cohort of corticosteroid-naïve patients. It appears to be well tolerated, with fewer safety concerns than typically seen with long-term standard-of-care corticosteroid treatment and lacking the stunting of growth that other approved corticosteroids cause. Further studies will directly compare vamorolone to prednisone and are expected to yield results consistent with those presented herein.

### Example 4: Phase 2b Clinical Trial in DMD

VISION-DMD is a pivotal Phase 2b study (VBP15-004) designed to demonstrate the efficacy and safety of vamorolone compared to placebo and prednisone (active control) for treating DMD. In the first 24-week double-blind period, 121 ambulant boys aged 4 to <7 years with DMD were randomized to receive vamorolone (low dose 2 mg/kg/day or high dose 6 mg/kg/day) or prednisone (0.75 mg/kg/day) or placebo. The second period of 24 weeks, where all participants receive vamorolone treatment on either of the two dose levels, will continue to capture additional longer-term safety and tolerability data.

The study met its primary endpoint of superiority in the change of time to stand from supine positioning to standing (TTSTAND) velocity with vamorolone 6 mg/kg/day versus placebo (p=0.002) with a treatment difference of 0.06 [95% CI: 0.02-0.10] rises/second from baseline. This result corresponded to a clinically relevant improvement in TTSTAND in the vamorolone 6 mg/kg/day group from 6.0 to 4.6 seconds and a corresponding deterioration in the placebo group from 5.4 to 5.5 seconds. The study also demonstrated superiority of vamorolone versus placebo across four of its secondary endpoints, including (in the order of pre-defined hierarchy): TTSTAND velocity for 2 mg/kg/day (p=0.02), 6MWT for 6 mg/kg/day (p=0.003) and 2 mg/kg/day (p=0.009), TTRW for 6 mg/kg/day (p=0.002). Any number p<0.05 is considered significant. There were no statistically significant differences between vamorolone 6 mg/kg/day and prednisone across the above endpoints.

The study completion rate at 24 weeks was 94% (or 114 of 121 participants). Vamorolone at both doses of 2 and 6 mg/kg/day showed a favorable safety and tolerability profile. In the vamorolone groups, no grade 3 or higher treatment-emergent adverse events (TEAEs) or adverse events leading to study discontinuation were observed. The total number of TEAEs was lower in the vamorolone 2 mg/kg/day (events n=96) and 6 mg/kg/day (n=91) groups than prednisone (n=120). In a prespecified analysis of clinically relevant adverse events (moderate, severe, serious, or leading to discontinuation due to safety), vamorolone 6 mg/kg/day was significantly superior to prednisone (n=6 vs. n=19, p=0.02).

Vamorolone also did not show stunting of growth as reported with conventional corticosteroids, which this study validated. Vamorolone 6 mg/kg/day versus prednisone 0.75 mg/kg/day showed a significant difference in growth velocity (p=0.02). The change in height Z scores from baseline to Week 24 showed growth deceleration in the prednisone group, whereas the 6.0 mg/kg/day vamorolone group showed an increased growth rate (p=0.02). The vamorolone 2.0 mg/kg/day group showed overall stable height change Z scores, but this did not reach significance relative to the growth deceleration seen with prednisone. These 24-week data were consistent with the absence of stunting of growth safety concern of prednisone and deflazacort in Example 3.

Bone turnover biomarkers showed prednisone to strongly reduce all bone biomarkers (osteocalcin, PINP, and CTX) at Week 24. In contrast, vamorolone did not decrease bone biomarkers (p<0.001 for both vamorolone 2.0 mg/kg and 6.0 mg/kg vs. prednisone for all three biomarkers) (Table 9).

TABLE 9
Bone turnover biomarkers in double-blind study VBP15-004

| | Prednisone | | | | |
|---|---|---|---|---|---|
| | 0.75 mg | Vamorolone 2.0 mg | | Vamorolone 6.0 mg | |
| | (N = 24) | (N = 17) | | (N = 23) | |
| | Mean (SEM) | Mean (SEM) | Difference | Mean (SEM) | Difference |
| | change at | change at | (p-value) vs. | change at | (p-value) vs. |
| Parameter | Week 24 | Week 24 | prednisone | Week 24 | prednisone |
| Osteocalcin | −15.2 (2.7) | 8.3 (3.1) | 23.5 | 1.7 (2.8) | 16.9 |
| (ng/ml) | | | (p < 0.001) | | (p < 0.001) |
| Procollagen 1 N- | −134 (22) | 48 (27) | 182 | −11 (22) | 124 |
| Terminal | | | (p < 0.001) | | (p < 0.001) |
| Propeptide (P1NP) | | | | | |
| (ng/mL) | | | | | |
| Type I Collagen C- | −300 (43) | 177 (52) | 477 | 90 (44) | 390 |
| Telopeptides | | | (p < 0.001) | | (p < 0.001) |
| (CTX) (pg/mL) | | | | | |

Pre-specified AESIs typically for corticosteroids were higher in prednisone-treated subjects than vamorolone-treated subjects after 24 weeks of treatment (Table 10). This difference was driven by a higher incidence of behavior problems in prednisone-treated subjects (32.3%) compared to vamorolone-treated subjects (16.7% and 21.4% in the 2.0 and 6.0 mg/kg dose groups, respectively) (Table 11). In addition, moderate/severe behavior problems were reported in 22.6% of subjects in the prednisone group, compared to 1.7% of vamorolone-treated subjects. Vamorolone also showed a superior safety profile compared to prednisone for clinically relevant TEAEs, prospectively defined as TEAEs of at least moderate severity, serious AEs, or TEAEs leading to treatment discontinuation (Table 10). This also reflects the difference between vamorolone and prednisone in behavior-related AEs, with clinically relevant psychiatric events reported by 19.4% of prednisone-treated subjects compared to no vamorolone-treated subjects. Of note, this clear point of difference between vamorolone and prednisone emerged after only 24 weeks of treatment; based on trends seen for other AESIs within this short period, it can be expected that additional clinical safety differences may be seen after longer vamorolone treatment periods in this study when compared to the corticosteroid-treated cohort in the FOR-DMD study.

TABLE 10
Summary of adverse events in double-blind study VBP-004

| | Placebo | PDN 0.75 | VAM 2.0 mg | VAM 6.0 mg |
|---|---|---|---|---|
| | (N = 29) | mg (N = 31) | (N = 30) | (N = 28) |
| Event type | n (%); F | n (%); F | n (%); F | n (%); F |
| All TEAEs | 23 (79.3); 77 | 26 (83.9); 120 | 25 (83.3); 96 | 25 (89.3); 91 |
| Severe TEAEs | — | 1 (3.2); 1 | — | — |
| (CTCAE Grade ≥3) | | | | |
| Deaths | — | — | — | — |
| Serious adverse | — | — | 1 (3.3); 1 | — |
| events | | | | |
| TEAEs leading to | — | 1 (3.2); 1 | — | — |
| discontinuation | | | | |
| All AESIs1 | 20 (69.0); 45 | 24 (77.4); 69 | 20 (66.7); 49 | 22 (78.6); 52 |
| AESIs¹ of at least | 5 (17.2); 5 | 11 (35.5); 15 | 2 (6.7); 5 | 1 (3.6); 1* |
| moderate severity | | | | |
| Clinically relevant | 9 (31.0); 9 | 13 (41.9); 19 | 8 (26.7); 11 | 4 (14.3); 6* |
| AEs2 | | | | |

PDN = prednisone;
VAM = vamorolone;
TEAE = treatment-emergent adverse event;
AESI = adverse event of special interest;
n (%) represents number and percentage of subjects reporting one or more events;
F = frequency of adverse events (AE count)
¹Based on pre-defined MedDRA search criteria for 11 AESI categories (behavior problems, blood glucose related problems, gastrointestinal symptoms, increased arterial blood pressure, immune suppression/infections, skin/hair changes, cataracts/glaucoma, cushingoid features, weight gain, bone fractures, slow growth)
2Adverse events of at least moderate severity, serious adverse events and adverse events leading to discontinuation
*Statistically significant difference (p < 0.05) vs. prednisone in hazard ratio based on proportional means regression models for recurrent events, i.e., allowing multiple events for each subject

TABLE 11
Behavior adverse events of special interest (AESI)¹
in double-blind study VBP15-004

| | | | VAM 2.0 mg | | VAM 6.0 mg | |
|---|---|---|---|---|---|---|
| | PDN 0.75 mg | | (N = 30) | | (N = 28) | |
| | (N = 31) n (%); F | | n (%); F | | n (%); F | |
| AESI Group | Any | Moderate/ | Any | Moderate/ | Any | Moderate/ |
| Preferred Term | severity | severe | severity | severe | severity | severe |
| Any Behavior | 10 (32.3); 16 | 7 (22.6); 8 | 5 (16.7); 6 | 1 (3.3); 1 | 6 (21.4); 9 | — |
| AESI | | | | | | |
| Abnormal | 2 (6.5); 2 | 1 (3.2); 1 | 2 (6.7); 2 | — | 1 (3.6); 1 | — |
| behavior | | | | | | |
| Aggression | 2 (6.5); 3 | 2 (6.5); 2 | — | — | 1 (3.6); 1 | — |
| Agitation | — | — | — | — | 1 (3.6); 1 | — |
| Anger | 1 (3.2); 1 | — | — | — | — | — |
| Anxiety | — | — | — | — | 1 (3.6); 1 | — |
| Emotional | 1 (3.2); 1 | 1 (3.2); 1 | — | — | — | — |
| disorder | | | | | | |
| Irritability | 1 (3.2); 1 | — | — | — | 3 (10.7); 3 | — |
| Mood altered | — | — | — | — | 1 (3.6); 1 | — |
| Mood swings | 1 (3.2); 2 | 1 (3.2); 1 | — | — | — | — |
| Sleep disorder | 1 (3.2); 1 | 1 (3.2); 1 | — | — | 1 (3.6); 1 | — |
| Initial insomnia | 1 (3.2); 1 | — | — | — | — | — |
| Personality | 1 (3.2); 1 | 1 (3.2); 1 | — | — | — | — |
| change | | | | | | |
| Poor quality sleep | — | — | 1 (3.2); 1 | — | — | — |
| Psychomotor | 3 (9.7); 3 | 1 (3.2); 1 | 2 (6.7); 2 | — | — | — |
| hyperactivity | | | | | | |
| Skin laceration | — | — | 1 (3.3); 1 | 1 (3.3); 1 | — | — |

PDN = prednisone;
VAM = vamorolone;
n (%) represents the number and percentage of subjects reporting one or more events;
F = frequency of adverse events (AE count)
¹Based on a pre-defined search of MedDRA terms, as defined in the statistical analysis plan for study VBP-004

In summary, for efficacy, vamorolone was significantly superior compared to placebo on the primary and four of the secondary outcomes. Bone loss caused by the corticosteroid class can predispose DMD pediatric patients to vertebral and long bone fractures, stunting of growth, bone fragility, and osteopenia. These effects impact the quality of life and may cause discontinuation of corticosteroid treatment with the resulting progression of the disease. Preliminary evidence also suggests that vamorolone has an improved safety profile on behavioral adverse events relative to corticosteroids.

These data also showed that vamorolone was effective over a three-fold range of doses, between 2 mg/kg/day to 6 mg/kg/day. This range permits physicians to prescribe, for example, an initial dose of 6 mg/kg/day and down titrate to a dose below 6 mg/kg/day and down to 2 mg/kg/day. Safety concerns were also improved compared to corticosteroids. Thus, vamorolone fulfills an unmet medical need for treating DMD as it provides statistically significant and clinically meaningful efficacy on motor outcomes vs. placebo with comparable efficacy to prednisone, but without the severe bone morbidities that limit treatment with corticosteroids. Vamorolone will spare DMD boys from bone morbidities and potentially behavioral problems for the corticosteroid class.

### Example 5

A metanalysis of ACTH stimulation reports from 1966 to 2006 (Kazlauskaite et al. “Corticotropin tests for hypothalamic-pituitary-adrenal insufficiency: a metaanalysis.” 93 JOURNAL OF CLINICAL ENDOCRINOLOGY & METABOLISM 4245-4253 (2008)), suggested screening the basal cortisol (e.g., pre-test levels) from 8-10 am dividing the patients into three groups:
<138 nmol/L (>92% chance of adrenal insufficiency)
138-365 nmol/L (40% chance of adrenal insufficiency)
>365 nmol/L (<9% chance of adrenal insufficiency)

These same criteria were used in the Phase 2 clinical trial for treating Duchenne muscular dystrophy (DMD) with vamorolone. Any DMD patients with a basal cortisol level of less than 138 nmol/L were labeled as “low,” indicating a greater than 92% chance of having adrenal insufficiency. When applied to the 48 successful ACTH tests in the placebo group from the Phase 2 trial, the patients fell into three groups:
<138 nmol/L (>92% chance of adrenal insufficiency). n=12; 25%
138-365 nmol/L (40% chance of adrenal insufficiency). n=34; 71%
>365 nmol/L (<9% chance of adrenal insufficiency). n=2; 4%

These data are consistent with the peak cortisol at 30 plus 60 minutes, showing that these DMD patients expressed adrenal insufficiency at baseline.

The normative pediatric data for ACTH challenge tests for four to seven-year-old children have a mean of about 900 nmol/L. The mean ACTH levels for the baseline and placebo groups in the DMD study was about 550 nmol/L. This alternative analysis has shown that DMD patients have intrinsic adrenal insufficiency at baseline.

In clinical practice guidelines for adrenal insufficiency published the by Endocrine Society (https://www.endocrine.org/clinical-practice-guidelines/primary-adrenal-insufficiency, last accessed incorporated), the standard of care diagnosis and treatment of adrenal insufficiency is that “patients should undergo a blood test to measure levels of adrenocorticotropic hormone (ACTH)—the hormone that signals the adrenal glands to produce cortisol—to establish a primary adrenal insufficiency diagnosis.” The Endocrine Society has further advised that “patients who have a confirmed diagnosis of primary adrenal insufficiency should undergo glucocorticoid replacement therapy-typically with hydrocortisone (cortisol), the glucocorticoid hormone naturally produced by the adrenal glands.” In some embodiments, the symptoms of adrenal insufficiency are chosen from extreme fatigue, muscle weakness, weight loss, decreased appetite, and slow growth.

Vamorolone effectively treated adrenal insufficiency and its symptoms in DMD patients. Fatigue and weakness significantly improved in five motor outcomes tested. Vamorolone effectively reduced weight loss and increased appetite in DMD children. Vamorolone also improved growth rates in DMD children. Thus, vamorolone has shown effects consistent with effective treatment of adrenal insufficiency.

### Embodiments

The detailed description set forth above is provided to aid those skilled in the art in practicing the present disclosure. However, the disclosure described and claimed herein is not to be limited in scope by the specific embodiments herein disclosed because these embodiments are intended as an illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

What is claimed is:

1. A method of treating or reducing the symptoms of congenital adrenal hypoplasia in a human patient, comprising administering to the human patient in need thereof a therapeutically effective amount of a compound having the structural formula 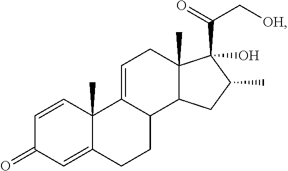 [SMILES: C[C@@H]1CC2C3CCC4=CC(=O)C=C[C@]4(C)C3=CC[C@]2(C)[C@@]1(O)C(=O)CO]
or a salt or polymorph thereof.

2. The method of claim 1, wherein the symptoms are chosen from weakness, fatigue, loss of appetite, weight loss, vomiting, difficulty with feeding, dehydration, hypoglycemia, low sodium levels, and shock.

3. The method of claim 1 or 2, wherein the human patient also has muscular dystrophy

4. The method of claim 3, wherein the muscular dystrophy is chosen from Duchenne muscular dystrophy and Becker muscular dystrophy.

5. The method of claim 4, wherein the muscular dystrophy is Duchenne muscular dystrophy.

6. The method of any one of claims 3-5, wherein the treatment is characterized by an increased velocity for time run/walk ten meters (TTRW).

7. The method of claim 6, wherein the TTRW velocity increased by at least 0.3 meters per second.

8. The method of any one of claims 3-7, wherein the treatment is characterized by an increased velocity for time to climb four stairs (TTCLIMB).

9. The method of claim 8, wherein the TTCLIMB velocity increased by at least 0.05 stairs per second.

10. The method of any one of claims 1-9, without increasing the incidence of vertebral fractures in the human patient.

11. The method of any one of claims 1-10, without increasing the incidence of behavior adverse events in the human patient.

12. The method of claim 11, wherein the behavior adverse event is chosen from one or more of aggression, agitation, anger, emotional disorder, irritability, mood swings, sleep disorder, initial insomnia, and personality change.

13. The method of claim 12, wherein the behavior adverse event is chosen from one or more of anger, mood swings, and personality change.

14. The method of any one of claims 11-13, wherein the patient is assessed with a Pediatric Anxiety Rating Scale (PARS) III questionnaire.

15. The method of any one of claims 1-14, without decreasing lean body composition and bone density in the human patent.

16. The method of claim 15, wherein the body composition and bone density are measured via dual-energy X-ray absorptiometry (DXA).

17. The method of any one of claims 1-16, wherein the human patient's body composition is leaner than in the human patient taking a therapeutically effective amount of hydrocortisone for congenital adrenal hypoplasia.

18. The method of any one of claims 1-17, wherein the human patient's bone density is greater than in the human patient taking a therapeutically effective amount of hydrocortisone for congenital adrenal hypoplasia.

19. The method of any one of claims 1-18, wherein total body lean mass index of the human patient showed greater positive changes in the human patient who has taken a therapeutically effective amount of hydrocortisone for congenital adrenal hypoplasia.

20. The method of any one of claims 1-19, wherein the rate of osteoporosis in the human patient is less than in the human patient taking a therapeutically effective amount of hydrocortisone for congenital adrenal hypoplasia.

21. The method of any one of claims 15-20, the difference between chronological age of the human patient and the bone age of the human patient is reduced.

22. The method of any one of claims 1-21, wherein the human patient demonstrates reduced positive transcriptional activity.

23. The method of any one of claims 1-22, wherein administration is for at least 6 months.

24. The method of claim 23, wherein the administration is for at least 12 months.

25. The method of claim 24, wherein the administration is for at least 18 months.

26. The method of claim 25, wherein the administration is for at least 24 months.

27. The method of claim 26, wherein the administration is for at least 30 months.

28. The method any one of claims 1-27, wherein the months are consecutive.

29. The method any one of claims 1-27, wherein the months are cumulative.

30. The method any one of claims 1-22, wherein between about 1 mg/kg/day and about 12 mg/kg/day of the compound is administered.

31. The method of claim 30, wherein between about 2 mg/kg/day and about 6 mg/kg/day of the compound is administered.

32. The method of claim 31, wherein about 2 mg/kg/day of the compound is administered.

33. The method of claim 32, wherein the administration of 2 mg/kg/day of the compound has a decreased risk of weight gain for the human patient.

34. The method of claim 31, wherein about 6 mg/kg/day of the compound is administered.

35. The method of any one of claims 1-34, wherein the human patient is 1 day to 18 years old.

36. The method of claim 35, wherein the human patient is between 2 and 18 years old.

37. The method of claim 36, wherein the human patient is between 4 and 12 years old.

38. The method of claim 37, wherein the human patient is between 4 and 7 years old.

39. The method of any one of claims 1-38, wherein the human patient is male.

40. The method of any one of claims 1-39, wherein the compound is administered orally.

41. The method of any one of claims 1-40, wherein the compound is administered as a solution or suspension.

42. The method of claim 41, wherein the solution or suspension comprises about 4 wt. % of the compound.

43. The method of claim 41 or 42, wherein the solution or suspension further comprises a flavoring agent.
